(19) United States
(12) Patent Application Publication
(10) Pub. No.: US 2008/0138399 A1
(43) Pub. Date: Jun. 12, 2008

(54) DUAL RELEASE NICOTINE FORMULATIONS, AND SYSTEMS AND METHODS FOR THEIR USE

(75) Inventor: Igor Gonda, Hayward, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Aradigm Corporation

(21) Appl. No.: 11/931,921

(22) Filed: Oct. 31, 2007

Related U.S. Application Data
(63) Continuation-in-part of application No. 11/097,598, filed on Apr. 1, 2005.
Application No. 11/097,598 is a continuation of application No. 10/913,103, filed on Aug. 6, 2004, now Pat. No. 6,874,507.
Application No. 10/913,103 is a division of application No. 10/147,390, filed on May 15, 2002, now Pat. No. 6,799,576.
Application No. 10/147,390 is a continuation-in-part of application No. 09/611,423, filed on Jul. 7, 2000, now abandoned.
(60) Provisional application No. 60/982,070, filed on Oct. 23, 2007.
(60) Provisional application No. 60/868,238, filed on Dec. 1, 2006.
(60) Provisional application No. 60/911,044, filed on Apr. 10, 2007.
(60) Provisional application No. 60/913,185, filed on Apr. 20, 2007.
(60) Provisional application No. 60/916,510, filed on May 7, 2007.
(60) Provisional application No. 60/917,190, filed on May 10, 2007.
(60) Provisional application No. 60/144,140, filed on Jul. 16, 1999.

(51) Int. Cl. A61K 31/465; A61K 9/127; A61K 9/12; A61P 43/00

(52) U.S. Cl. 424/450; 514/343; 424/46; 424/45

(57) ABSTRACT

This invention relates generally to a method to provide habitual tobacco users with products, methods and apparatus to reduce and eventually terminate their dependence on nicotine containing products. More specifically, the invention relates to a nicotine-based medicament that is formulated in such a way as to effectively reduce or eliminate the sensations of craving associated with addictive nicotine use.

### PRIORITY DOCUMENTS

This application claims the benefit of U.S. Provisional Application Nos. 60/982,070, filed Oct. 23, 2007; 60/868,238, filed Dec. 1, 2006; 60/911,044, filed Apr. 10, 2007; 60/913,185, filed Apr. 20, 2007; 60/916,510, filed May 7, 2007; and 60/917,190, filed May 10, 2007 and is a continuation-in-part of U.S. application Ser. No. 11/097,598 filed on Apr. 1, 2005 which is a continuation of Ser. No. 10/913,103, filed Aug. 6, 2004, issued Apr. 5, 2005, as U.S. Pat. No. 6,874,507, which is a divisional of 10/147,390, filed May 15, 2002, issued Oct. 5, 2004, as U.S. Pat. No. 6,799,576, which is a continuation-in-part of Ser. No. 09/611,423, filed Jul. 7, 2000, now abandoned, which claims benefit to U.S. Provisional Application No. 60/144,140 filed on Jul. 16, 1999, all of which applications are incorporated herein by reference.

### FIELD OF THE INVENTION

This invention relates generally to compositions and methods allowing habitual smokers to reduce and eventually terminate their dependence on nicotine containing products, particularly tobacco products. More specifically, the invention relates to a nicotine-based medicament that is formulated in such a way as to effectively reduce or eliminate the sensations of craving associated with addictive smoking behavior.

### BACKGROUND

The use of tobacco products presents a critical international public health problem. Addiction to nicotine represents an enormous health, social, and financial burden. Cigarettes are among the most addictive products known, and a vast majority of people who attempt to quit smoking relapse within days (Henningfield, 1991). They are the world's leading cause of preventable death, contributing to 5 million premature deaths in 2000, and is estimated to increase to 10 million by 2020 (Nides, 2006). In the United States, fewer than 10% of the nearly 20 million people who quit smoking remain abstinent one year later. Thus, only 2-3% of smokers become non-smokers each year (Henningfield, 1995).

Nicotine is the primary active ingredient in cigarettes that reinforces individual smoking behavior. Nicotine interacts with nicotinic cholinergic receptors in the brain to induce the release of neurotransmitters and produce an immediate reward—the “rush” that smokers experience—that is associated with a rapid rise in blood level. A persistent stimulus is also produced, and is associated with a high blood level of nicotine. As such, the dopaminergic reward system is activated by nicotine which eventually results in nicotine dependency. However, it is the other constituents of tobacco and not nicotine that cause widespread mortality and morbidity.

Nicotine replacement therapies (NRTs) are pharmacological nicotine delivery systems developed to improve outcomes in tobacco cessation treatment. Abrupt cessation of tobacco use often produces a withdrawal syndrome that includes depression and/or anxiety, hunger, sleep disruption, and inability to concentrate (Hughes, 1986). Withdrawal symptoms usually peak within a few days of cessation and can last for up to 4 weeks. More than half the smokers who quit will relapse within one week, coinciding with the peak in withdrawal symptoms (Henningfield, 1995).

NRTs help with smoking cessation by reducing the severity of withdrawal symptoms, uncoupling the behavioral changes needed to quit from the unpleasant effects of nicotine withdrawal, thereby partially enhancing mood and improving concentration.

There are now a number of approved nicotine-containing smoking cessation products available by prescription starting with the launch of a gum in 1984 and with OTC (over-the-counter) products available since about 1996. Smoking cessation products in the U.S. are available in gums, patches, lozenges, and an inhaler and a nasal spray and use either nicotine or nicotine derivatives.

Nicorette® gum (nicotine polacrilex) was approved for prescription sale in 1984, and the FDA began allowing its sale without a prescription in February 1996. The nicotine patch (containing nicotine free base), also known as a nicotine transdermal system, has been available in the US by prescription since 1991 and by OTC since July 1996. It is sold under the brand names Nicoderm®, Nicotrol®, Habitrol®, and Prostep®. The first inhaled dosage form of nicotine, Nicotrol NS®, designed to be used as a nasal inhaler, was launched in September 1996. FDA approved the Nicotrol® nicotine inhalation system (containing nicotine free base) for smoking cessation in May 1997. Nicotine enters the user's mouth through a mouthpiece attached to a plastic cartridge. Although termed an “inhaler,” it does not deliver nicotine to the lungs the way a cigarette does. Almost all of the nicotine travels only as far as the mouth and throat, where it is absorbed through the mucous membranes (Schneider, 2001). Nicotrol® nicotine nasal spray was approved in March 1996 for sale by prescription. The nicotine is inhaled into the nose from a pump bottle and absorbed through the nasal lining into the bloodstream. Each form of NRT has its own advantages and limitations.

Obtaining nicotine from NRT is considerably safer than doing so from cigarettes, as the user is not exposed to any of the myriad harmful compounds of tobacco combustion. Although long-term use of NRT is not thought to be associated with any serious harmful effects (Molyneux, 2004), the current forms of NRT smoking cessation products have very poor efficacy; a recent survey has suggested that the percentage of abstinent smokers varies between 14-24% for the various NRTs (Silagy, 2004). No existing NRT offers the pharmacokinetic properties that adequately satisfy the subjective craving that smokers experience and which would allow smokers to easily transition from a tobacco product to a safer NRT product from which he or she may begin to gradually reduce his or her dependence toward eventual cessation of all nicotine and tobacco containing products.

Low nicotine replacement levels or under-dosing and the inability to adequately satisfy a smoker's craving is likely a significant factor leading to the failure of many NRTs: “Dependence on smoking appears to be related, at least in part, to the achievement of a rapid rise in plasma nicotine concentrations. If this assessment is correct, the most desirable adjuvant for smoking cessation would be one that closely mimics this pattern of plasma nicotine concentrations” (Svensson, 1987). Optimal nicotine replacement for smoking cessation may initially require both reproducing and then sustaining the nicotine levels in the blood stream and in the brain that are produced by habitual cigarette smoking so that nicotine withdrawal symptoms are minimized while the behavioral aspects of smoking are modified towards cessation (Russell, 1986; Perkins, 1986). The most important factor in successful smoking cessation may be the ability to approximate the plasma nicotine concentration pharmacokinetics obtained with cigarettes in order to satisfy the subjective cravings. For example, in contrast to the currently available slow-release nicotine polacrilix gums (the nicotine is bound or complexed with the polacrilex resin), there is evidence that rapid-release nicotine gum reduces craving more rapidly (see Niaura et al, Addiction 2005 100; 1720-1730).

It is also difficult to reproduce the pharmacokinetic pattern of multiple nicotine concentration peaks that are achieved by successive “puffs” from a cigarette, cigar, or pipe without the use of multiple doses. Thus, the slow rise and lack of achieving an adequate plasma nicotine concentration “steady state” around the time of self-administration suggests a pharmacokinetic explanation for the relatively high failure rate of some NRTs; administering one large dose equivalent to the total nicotine inhaled from a cigarette or other tobacco containing product could be dangerous and it would only provide a short duration of the high nicotine concentration in the blood stream. Providing smokers with nicotine in a form that does not require inhalation of tobacco smoke could be an effective way to avoid the hazards associated with smoking.

There are as yet no currently approved products that are able to achieve high peak nicotine concentrations combined with the benefits of providing a sustained, slow-release plasma level over a prolonged period of time. U.S. Pat. No. 5,935,604 describes a nasal drug delivery composition comprising nicotine or a pharmacologically-acceptable salt or derivative wherein the composition is adapted to delivery of a pulse of nicotine for rapid absorption and a controlled release of nicotine for subsequent sustained absorption. However, the small surface area of the nasal cavity does not afford the same opportunities as the large surface area of the respiratory tract with surfaces and anatomical locations varying in their absorption barrier properties.

There is a clear need for improvement in nicotine cessation treatment. Development of new systems is critical in an effort to both bypass limitations of existing systems and to provide effective options for matching smokers to treatment.

### SUMMARY

The present invention provides tobacco-less formulations and methods allowing a person to overcome a smoking habit. The invention provides the smoker with a combination dosage form that has readily bioavailable nicotine causing rapidly high “peak” nicotine levels that are thought to be associated with “craving” for nicotine, and a slow release component of the formulation that maintains the nicotine plasma levels over a prolonged period of time. The dual release nature of this formulation, the rapid release component to achieve a quick peak nicotine plasma concentration, and the slow-release component to achieve lower, sustained plasma concentrations, will allow the subjective cravings experienced by the smoker to be minimized. Such formulations could be administered by injection, or inhalation into the airways and lungs via mouth or nose, or applied to the nasal mucosa or swallowed or given transdermally (by a combination of e.g., microneedles plus a slow release patch), or via the buccal cavity, etc.

The rapid-release component could be nicotine, or a nicotine-like substance, or a salt of nicotine, in liquid form, or dissolved in a suitable solvent, or be in a rapidly soluble solid form. To achieve rapid absorption, a penetration enhancer may be added. The slow releasing component could be a liposomal formulation, or a cyclodextrin complex, or nicotine in a solid matrix slowly releasing (e.g., a polylacticglycolic acid microsphere, an ion-exchange resin such as polacrilex [Amberlite IRP64], or lipid-based microspheres). The mixture of the slow releasing and the rapidly-releasing components (e.g., a nicotine salt solution in water together with nicotine-loaded liposomes in the same aqueous formulation; or nicotine dissolved in a suitable propellant plus nicotine-containing slow-release particles dispersed in a propellant, the mixture being enclosed in a “metered dose inhaler”) would be administered into the body, e.g., by inhalation of an aerosolized mixture. Methods of formulating liquids and liquid inhalers are disclosed in U.S. Pat. Nos. 5,364,838; 5,709,202; 5,497,763; 5,544,646; 5,718,222; 5,660,166; 5,823,178; and 5,910,301; all of which are incorporated by reference to describe and disclose such. Formulations for both rapid and slow release of nicotine include aqueous formulations, aqueous saline formulations, and ethanol formulations. A dry powder formulation comprising a pharmacologically acceptable salt of nicotine alone or with additives such as components to prevent the particles from sticking together may be used.

All of these formulations may be included with additional components such as permeation enhancers, buffers, preservatives and excipient and carrier components and additives normally included within formulations for aerosolized drug delivery.

The advantages of this nicotine product would be that a single administration would achieve a safe and effective peak of nicotine in the blood stream and then maintain the concentration of nicotine at a suitable level for prolonged period of time to avoid “craving” for tobacco products. This combination product could be used both as a smoking cessation tool as well as a safer replacement for tobacco smoking.

In addition, an aspect of the invention is that varying the amounts of each of the dual release formulation components, makes it possible to gradually reduce the blood concentrations of nicotine. In this way, the smoker is able to achieve eventual freedom from dependence on nicotine and any nicotine containing tobacco product.

In one embodiment the invention provides a tobacco-less composition that has a pharmaceutically active nicotine formulation for delivery to a patient. The nicotine formulation has at least two forms of nicotine, a fast release form and a slow release form. At least the fast release form of nicotine is inhaled, and is present in an amount to provide a first form of nicotine arterial concentration in the patient within 5 minutes of delivery. The nicotine arterial concentration produced by the first nicotine form is preferably at least 10 ng/ml, but may be 15, 20, 25, 30, 35, 40, 50 or more ng/ml nicotine, being limited by the amount necessary to address the nicotine addiction while not reaching toxic levels.

The second form of nicotine is present in an amount to maintain a second form of nicotine arterial concentration in the patient for at least 60 minutes after delivery. This may be augmented by addition of slow release components such as cyclodextrin, encapsulation of the active nicotine, chemical or physical modification of the form of nicotine and the like. The arterial concentration of the second form of nicotine is at least 5 ng/ml, preferably 7, 10, 12, 15 or 20 ng/ml.

Encapsulation of the second form of nicotine may be by any method known in the art. For example, the nicotine form may be encapsulated in a microsphere, such as a polyglycolide microsphere, or a liposome. The form of nicotine may be further modified by optional addition of a bioadhesive component, such as hyaluronic acid. Encapsulation greatly enhances the variation in nicotine forms that may be utilized in the tobacco-less formulation. For example, by creating controlled microenvironments, encapsulation allows different pH forms, different salts and different compounds to be associated with one form of nicotine without contamination of the other. For example, by allowing each nicotine form to be optionally encapsulated, the tobacco-less formulation may include nicotine forms delivered at different pH values. This is important as the pH of the solution containing the compound determines whether the compound is a free base, acid or salt. It is well known in the art that free base nicotine is much more potent than salts in eliciting a nicotine response in humans. Thus by delivering the first form of nicotine at a basic pH and the second form of nicotine at an acidic pH augments the invention in providing a greater effective nicotine in the initial nicotine bolus while also augmenting the slow release aspect of the second nicotine form.

In certain embodiments, the first and second forms of nicotine may also be packaged and/or delivered to the patient separately. These embodiments are distinct from those having the first and second forms of nicotine present in the same tobacco-less formulation. Packaging the two forms of nicotine separately allows the first form to be inhaled or administered via another route affording an initial pulse of nicotine to the arterial circulation, with the second form being delivered orally, transdermally or via some route consistent with the slow release nature of the second form. Lozenges, gums and quick dissolve strips are just some examples of compositions suitable for oral administration, with more examples presented below.

Pharmaceutically active nicotine formulations of the invention may be creams, gels, solids, patches, lozenges, gums, fast dissolving strips, powders, liquids, suspensions, emulsions and the like. In some embodiments the nicotine formulation is suitable for forming an aerosol. Such aerosols may include a propellant or be driven by mechanical manipulation without inclusion of a propellant. With aerosolized embodiments of the claimed invention the first form of nicotine typically has a smaller particle diameter than the second form of nicotine. For example, the first form of nicotine may have a particle diameter between about 1 μm and about 4 μm, more preferably between about 2 or 3 μm in diameter. Second form of nicotine particles typically have a diameter between about 4 μm and about 12 μm

Tobacco-less formulations of the invention may also include antidepressant or anxiolytic compounds or other supplemental drugs, excipients or other compounds that enhance the formulation chemically, pharmaceutically or in its ease or pleasure of use. In theory, the antidepressant or anxiolytic should allow the patient to make that final transition off nicotine entirely more easily.

The present invention also includes methods for treating a patient with tobacco-less nicotine formulation described above. The methods include delivering the nicotine formulation to a patient.

One aspect of the invention is a method of treatment, comprising:

(a) aerosolizing a formulation comprised of nicotine creating aerosolized particles which are sufficiently small to target and deposit predominantly in a particular lower area of the respiratory tract such as the alveoli. The particles targeting this area will have a relatively small size, e.g. 0.5 micron to about 2 microns in diameter.

(b) in the next step the patient inhales the aerosolized particles of (a) into the respiratory tract, preferably targeted to a specific area of the lower respiratory tract where the deposited particles cross into the patient's circulatory system.

In step (c), steps (a) and (b) are repeated a plurality of times. Specifically, the patient may repeat these steps any number of times such as every time the patient would normally smoke a cigarette. At this point the patient could continue the treatment protocol in this manner and gradually decrease the number of times the patient administers aerosolized nicotine until the patient is no longer addicted to nicotine. Decreasing the amount of aerosolized nicotine could also be done by decreasing the concentration of nicotine within the aerosolized particles by decreasing the concentration of nicotine in the formulation and/or decreasing the size of the aerosolized dose.

Preferably the method of the invention continues with a step (d) which involves aerosolizing formulation comprised of nicotine in order to create aerosolized particles which are larger in size than the aerosolized particles produced in step (a). These larger particles are directed towards a particular area of the patient's respiratory tract, e.g. the mid-region of the patient's respiratory tract. (See FIGS. 1 and 2) These particles could have a size in the range of about 2 microns to about 4 microns.

In the following step (d) the patient inhales the aerosolized particles of (d) thereby targeting the particular desired area of the patient's respiratory tract such as the mid region. Thereafter, steps (d) and (e) are repeated a plurality of times. At this point the patient can decrease the amount of nicotine being delivered as indicated in the same manner as indicated above step (c). Alternatively, the method of the invention can be continued so that a third phase of treatment can be carried out which phase is similar to the two phases described above. In accordance with the above invention it is possible to carry out the treatment in any number of phases. It would be impractical to develop a system which attempts to target each of the 24 different areas of the lung as outlined in Table 1 and shown in FIG. 1. Further, regardless of the system used there would be some overlap between the different areas of the lung. Because it may not be practical to specifically design the particles so that they are all larger in each of the phases the formulations may be designed so that a certain percentage of the particles within each phase of delivery is larger than the particles in the preceding phase.

Methods of the invention also include a method for treating a patient with the pharmaceutically active, tobacco-less nicotine formulation described. The method includes delivering the nicotine formulation to a patient in a first dosage amount and determining the patient's craving for nicotine after administering the formulation. The patient's craving for nicotine may be determined using any method known in the art, preferably the Fagerstrom test as described below. A second dosage amount of the formulation may be optionally administered to the patient. This second dosage amount may be a larger or smaller amount of the therapeutically effective nicotine formulation than was administered in the first dosage amount.

Another embodiment of the method of the invention involves treating a patient with a pharmaceutically active, tobacco-less nicotine formulation. The method includes delivering the nicotine formulation discussed herein to a patient over a first period of time in a first dosage amount. The nicotine formulation is then delivered to the patient over a second period of time in a second dosage amount. The first and second periods of time may be any period of time including indefinitely, but is more typically between one week and two months, preferably is a time period within 1, 2, 3, 4 or 5 weeks. The second dosage amount may also vary, being greater or less than the first dosage amount, depending upon the patient's reaction to the first dose. In all methods dosage amounts may be ramped up from a low amount to a higher amount to adjust for patient sensitivity to the nicotine formulations of the invention. Thus a novel aspect of this method is to start the sensitive patient with a lower dosage formulation that allows the patient to use less of a tobacco product as a source of nicotine. As the patient becomes accustomed to using the nicotine formulations of the invention, the dosage amount may be increased thus allowing a reduction in tobacco use as a source of nicotine. In this manner the patient can be freed from the health hazards of tobacco quickly and efficiently without suffering the effects of nicotine withdrawal. Once the formulas of the invention have entirely replaced tobacco as a source of nicotine, the dosage amount of the formulations of the invention may be gradually reduced to address the nicotine addition.

In another embodiment of the invention the different groups of targets can be designed to target different groups of areas of the lung. Thus, for example, as shown in Table 1 the areas of the lung are broken down into six general areas, these six general areas or even three general areas could be targeted (See Table 1). The higher levels of the respiratory tract can be targeted using larger and larger particles.

TABLE 1
Subdivision of the Respiratory Tree

| Generation | Name |
|---|---|
| 0 | Trachea |
| 1 | Primary bronchi |
| 2 | Lobar bronchi |
| 3 | Segmental bronchi |
| 4 | Subsegmental bronchi |
| 5 | Small bronchi |
| ↓ | |
| 10 | |
| 11 | Bronchioles, primary and secondary |
| ↓ | |
| 13 | |
| 14 | Terminal bronchioles |
| ↓ | |
| 15 | |
| 16 | Respiratory bronchioles |
| ↓ | |
| 18 | |
| 19 | Alveolar ducts |
| ↓ | |
| 23 | |
| 24 | Alveoli |

### BRIEF DESCRIPTION OF THE DRAWINGS

FIG. 1 compares the arterial nicotine profiles produced for cigarettes and various nicotine replacement therapies. The data is adapted from Rigotti, N. A., NEJM vol. 346, No. 7, (February 2002).

FIG. 2 depicts the mean arterial plasma nicotine concentrations for 16 human patients.

FIG. 3 depicts the mean craving scores for 16 human patients.

FIG. 4 summarizes the modified Fagerstrom test for evaluating intensity of physical dependence on nicotine. Adapted with permission from Heatherton T F, Kozlowski L T, Frecker R C, Fagerström K O. The Fagerström test for nicotine dependence: a revision of the Fagerström Tolerance Questionnaire. Br J Addict 1991; 86:1119-27.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

An “antidepressant” refers to a substance that is used in the treatment of mood disorders, as characterized by various manic or depressive affects.

The term “anxiolytic” refers to any compound that has the effect of relieving anxiety.

An “aerosol” is a cloud of solid or liquid particles suspended in a gas. The particles may be formed from any suitable composition including, but not limited to a solid such as a powder, a liquid, a gel, a cream, a suspension, an emulsion, or a colloidal mixture. Alternatively, any semi-solid or semi-liquid may be used. Methods of forming aerosols from prepared compositions are well-known in the art and described herein below.

A “bioadhesive component” is one which aids the compound containing it in associating with biological tissue.

A “slow release component” is one which imparts the ability to dissolve, be absorbed, transported or broken down more slowly thereby allowing the compound containing the slow release formula to persist.

When nicotine enters the circulatory system of a human patient it is oxidized to cotinine within four to six hours. The present invention includes the administration of cotinine and other nicotine derivatives provided such derivatives do not result in unacceptable adverse effects

The term “nicotine” is intended to mean the naturally occurring alkaloid known as nicotine, having the chemical name S-3-(1-methyl-2-pyrrolidinyl)pyridine, which may be isolated and purified from nature or synthetically produced in any manner. This term is also intended to encompass the commonly occurring salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate, ethanesulfonate, benzenesulfonate, p-toluene sulfonate, camphorate and pamoate salts. Nicotine is a colorless to pale yellow, strongly alkaline, oily, volatile, hygroscopic liquid having a molecular weight of 162.23 and the formula:

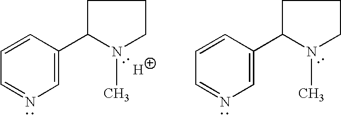

Structure and ionization of nicotine. Nicotine is approximately 10% of the particulate weight in cigarette smoke. Brand differences change this percentage. It is monoprotonated at most physiological pH values. The diprotonated ion would exist at pH values found in the stomach. Metabolism is largely due to oxidation. Cotinine is a major metabolite; however, there are at least 4 primary metabolites of nicotine and all are encompassed by the use of this term herein.

The term “form of nicotine” further includes any pharmacologically acceptable derivative, metabolite or analog of nicotine which exhibits pharmacotherapeutic properties similar to nicotine. Such derivatives and metabolites are known in the art, and include cotinine, norcotinine, nornicotine, nicotine N-oxide, cotinine N-oxide, 3-hydroxycotinine and 5-hydroxycotinine or pharmaceutically acceptable salts thereof. A number of useful derivatives of nicotine are disclosed within the Physician's Desk Reference (most recent edition) as well as Harrison's Principles of Internal Medicine. In addition, applicants refer to U.S. Pat. Nos. 5,776,957; 4,965,074; 5,278,176; 5,276,043; 5,227,391; 5,214,060; 5,242,934; 5,223,497; 5,278,045; 5,232,933; 5,138,062; 4,966,916; 4,442,292; 4,321,387; 5,069,094; 5,721,257; all of which are incorporated herein by reference to disclose and describe nicotine derivatives and formulations.

“Free base nicotine” refers to the form of nicotine that predominates at high pH levels. Free base nicotine is particularly potent and more addictive than nicotine salts which display a lower affinity to nicotinic receptors.

“A pharmaceutically active nicotine formulation” is a formulation having at least two forms of nicotine as components, and may include additional additives and drug dosages.

The physiologically active form of nicotine is the S-(−)-isomer. Certain compounds of the present invention may exist in particular geometric or stereoisomeric forms. The present invention contemplates all such compounds, including cis and trans isomers, R and S enantiomers, diastereomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

The term “dual-release” is used herein to refer to a formulation comprised of two components, one which releases nicotine or a nicotine derivative or nicotine substitute immediately, and one component which releases nicotine or a nicotine derivative or nicotine substitute over a prolonged period of time.

The term “diameter” is used herein to refer to particle size as given in the “aerodynamic” size of the particle. The aerodynamic diameter is a measurement of a particle of unit density that has the same terminal sedimentation velocity in air under normal atmospheric conditions as the particle in question. In connection with the present invention, it is important that particles, on average, have the desired diameter so that the particles can be inhaled and targeted to a specific area of the lungs. To target the alveolar ducts and alveoli the particles should have a diameter in a range of about 0.5μ to about 2μ.

The term “porous membrane” shall be interpreted to mean a membrane of material in the shape of a sheet having any given outer perimeter shape, but preferably covering a package opening which is in the form of an elongated rectangle, wherein the sheet has a plurality of openings therein, which openings may be placed in a regular or irregular pattern, and which openings have a diameter in the range of 0.25μ to 4μ and a pore density in the range of 1×10⁴ to about 1×10⁸ pores per square centimeter. The membrane functions to form an aerosolized mist when the formulation is forced through it. Those skilled in the art may contemplate other materials which achieve this function as such materials are intended to be encompassed by this invention.

The terms “treatment”, “treating”, and the like are used interchangeably herein to generally mean obtaining a desired pharmacological and/or physiological effect. The terms are used in a manner somewhat differently than the terms are typically used in that what is intended by the method of treatment of the invention is to allow a patient to overcome an addiction to nicotine and thereby allow the patient to quit smoking. The treating effect of the invention provides a psychological effect in that the invention originally delivers high doses of nicotine in a manner that simulates the nicotine delivery obtained from a cigarette. The patient then becomes accustomed to relying on the methodology of the invention to provide an immediate “rush” of nicotine. Thereafter, the particles of the aerosol are made larger. This prevents the particles from penetrating deeply into the lung and, therefore, to some extent, diminishes the “rush” of nicotine. However, the same amount of nicotine is still given to the patient in order to satisfy the overall nicotine craving. Eventually, the treatment of the invention reduces the amount of nicotine so as to allow the patient to completely “wean” off of nicotine and to quit smoking.

All publications mentioned herein are incorporated herein by reference to described and disclose specific information for which the reference was cited in connection with. The publications discussed herein are provided solely for their stated disclosure prior to the filing date of the present application. Nothing herein is to be construed as an admission that the invention is not entitled to antedate such publications by virtue of prior invention. Further, the actual publication date may be different from that stated on the publication and as such may require independent verification of the actual publication dates.

### DETAILED DESCRIPTION OF EXEMPLARY EMBODIMENTS OF THE INVENTION

### I. Introduction

The present invention provides a novel formulations and methods for their use that pharmacologically mimic the delivery of nicotine produced by smoking a cigarette. These formulations may be delivered in a single dose, such as a single breath from an inhaler, and provide an effective means for addressing and potentially eliminating a person's addiction to tobacco products including those used for smoking, chewing and sniffing. This nicotine dosage is completely tobacco-free and thus provides the patient an opportunity to address tobacco use and an addiction to nicotine individually. The separation of the habitual use of tobacco and nicotine addiction eases withdrawal from the dangerous habit and is believed to increase the opportunity to succeed in breaking the habit.

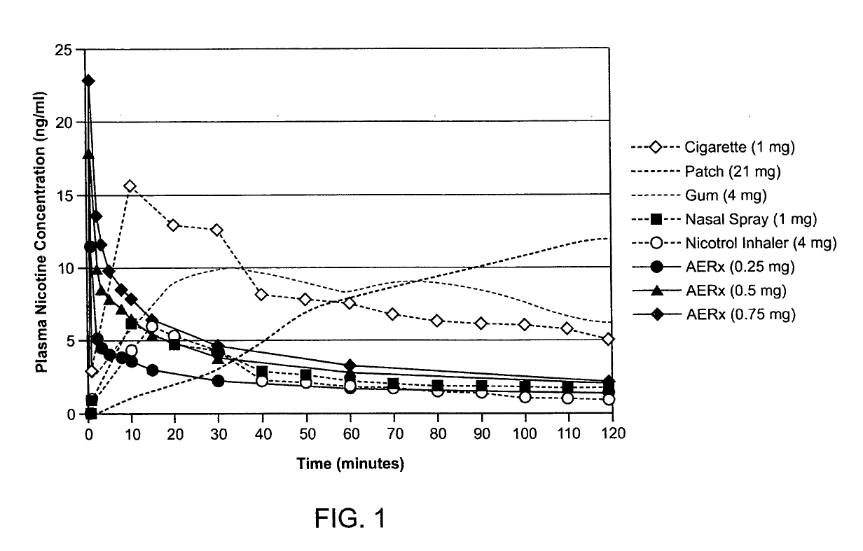

The formulations of the claimed invention contain at least two forms of nicotine that in combination mimic the pharmacological delivery of nicotine produced by smoking a cigarette without exposing the user to tobacco products. The formulas of the invention may be administered in any suitable manner known in the art that allows the pharmacological dosage pattern for nicotine described herein for the invention. Typically delivery will be by inhalation or sniffing the product into the respiratory tract, including the deep lung alveoli. In this manner the invention provides a nicotine dosage that rapidly peaks and then trails off maintaining arterial nicotine concentrations that mirror those produced by smoking tobacco (FIG. 1). The invention produces the nicotine dosage pattern by providing at least two forms of nicotine. The first form of nicotine includes sufficiently small particles that may be inhaled deeply into the lung, i.e. 50% or more of the particles are inhaled deeply into the lung and thereby quickly enter the patient's circulatory system. The invention also provides a second form of nicotine that is in a slow release form. This second form of nicotine ensures that the patient's arterial plasma nicotine concentration is maintained at levels that minimize craving for nicotine over a more extended time period.

The two forms of nicotine are typically dispersed in the form of particles that may originate from dry powder, liquid suspension or emulsion, microspheres suspended in an aqueous solution or dried, or any other physical manifestation that can be aerosolized allowing the particles to reach the intended region of the lung. By controlling the physical and chemical characteristics of the particles the release of the nicotine formulation to a patient's circulatory system as described herein may be controlled. The first and second forms of nicotine may be present in the formulations of the invention in different physical forms. For example, the formulation of the invention may be a liquid suspension of microspheres where the first form of nicotine is dissolved in the fluid component and the second form of nicotine is encapsulated in the microspheres. Alternatively, the formulation could be a dry powder where the first and second forms of nicotine are distinguished by particle size, roughness, diameter, composition or any combination of differences. A third exemplary formulation would be a heterogeneous suspension of microspheres where the first and second forms of nicotine are encapsulated in separate microsphere populations.

Particles suitable for use in the instant invention may be fabricated with the appropriate material, surface roughness, diameter and density for localized delivery to selected regions of the respiratory tract such as the deep lung, central or upper airways. For example, to increase the aerodynamic diameter, higher density or larger particles may be used for upper airway delivery or, smaller or lower density, e.g., porous particles, may be utilized for deep lung deposition. More preferably a mixture of different sized particles in a formulation may be administered to target different regions of the lung in one administration. Particles with degradation and release times ranging from seconds to hours can be designed and fabricated, based on factors such as the particle material. Techniques for fabricating such particles are well known in the art. For example, particles of the invention may be a form of nicotine that is a dry powder manufactured from nicotine with additional, optional, materials added to the formulation to impart desired characteristics as described in more detail below.

The present invention is also advantageous in that the rate at which the delivered nicotine enters the circulatory system can be gradually modulated, for example by gradually increasing the size of the aerosolized particles delivered to the patient leading to deposition in the parts of the respiratory tract from which absorption is slower. This can be done over any desired period of time and in any desired number of phases.

Moreover, the invention provides a means whereby the amount of nicotine delivered to the patient may be gradually decreased in a number of different ways. For example, nicotine delivery may be decreased by decreasing the concentration of nicotine in the tobacco-free formulation; nicotine may be decreased by decreasing the number of doses taken by the patient over a given period of time; nicotine may also be decreased by decreasing the size of the dose administered to the patient; and finally, nicotine delivery may be decreased by altering the nature of the formulation, as described herein.

As depicted in FIG. 1, current nicotine therapies are characterized by slow absorption and low blood levels of nicotine, limiting their utility. The present invention replaces the nicotine that a patient receives from using a tobacco product by providing a rapid pulse of bioavailable nicotine to the patient, followed by a slow release of nicotine providing a prolonged circulating concentration of nicotine. More specifically, the present invention provides a treatment methodology wherein a patient's initial arterial nicotine plasma concentration over a selected time, i.e., the arterial nicotine plasma concentration-rate profile, substantially correlates to that of the patient when smoking a cigarette; the slow release component of the formulation then maintains a minimum level of circulating nicotine over a longer period of time, in the range of 1 to 24 hours.

One treatment methodology of the present invention creates an aerosol of nicotine particles. As noted previously, the nicotine particles may be in powder form, or formed initially as droplets from any liquid containing nicotine including a solution or suspension of nicotine and aerosolized in any known manner including (1) moving the formulation through a porous membrane in order to create particles or (2) a dry powder where the particles of powder have been designed to have a desired diameter. By increasing the size of the particles from about 1-2 microns (μm) upwards causes the particles to be deposited higher in the respiratory tract. Without limiting the scope of the invention, it is generally known that higher regions of the respiratory tract have less tissue surface area than lower regions. As the rate of particle absorption is known to be directly proportional to the surface area of the tissue on which the particles are deposited, nicotine is absorbed more slowly through the mucosal membranes of the upper respiratory tract. Thus the effect of increasing particle size is to deposit the inhaled particle in a higher region of the respiratory tract with concomitant reduced absorption rate over time and a more sustained drug profile. Of course other mechanisms may also play a significant role in the release of nicotine to the circulatory system, and the present invention does not exclude such mechanisms. For example, clearance of larger particles from the upper respiratory tract may result in transport of those forms of nicotine to alternative locations and/or may contribute to the delay or sustained release of the nicotine form to the circulatory system.

Thus one method of practicing the present invention is to provide a formulation comprising two forms of nicotine, a first form characterized by fine particles of small diameter and a second form characterized by larger particles. The larger particles deposit in the upper respiratory tract providing low level sustained drug release, while the smaller particles penetrate to the deep lung providing a rapid pulse of available nicotine similar to that provided by a cigarette.

The method of the invention has applicability to smokers and users of other tobacco products wishing to quit or trying to quit who have experienced all or any of the nicotine withdrawal symptoms associated withdrawal from tobacco products. These symptoms include craving for nicotine, irritability, frustration or anger, anxiety, drowsiness, sleep disturbances, impaired concentration, nervousness, restlessness, decreased heart rate, increased appetite, and weight gain among others.

While particularly applicable to addressing habitual use of tobacco products, pulmonary, oral, or parenteral administration of nicotine could be of value for the treatment of other diseases, such as for patients suffering from neurodegenerative diseases, psychiatric disorders and other central nervous system disorders responsive to nicotinic receptor modulation (see U.S. Pat. Nos. 5,187,169; 5,227,391; 5,272,155; 5,276,043; 5,278,176; 5,691,365; 5,885,998; 5,889,029; 5,914,328). Such diseases include, but are not limited to, senile dementia of the Alzheimer's type, Parkinson's disease, schizophrenia, obsessive-compulsive behavior, Tourette's Syndrome, depression, attention deficit disorder, myasthenia gravis and drug addiction. These embodiments and others are discussed in greater detail, below. See Masterson (1991) U.S. Pat. No. 5,069,904; Wesnes and Warburton (1984) Psychopharmacology 82:147-150; and Warburton et al. (1986) Psychopharmacology 89:55-59.

### II. Tobacco-Less Formulations

Tobacco-less formulations of the present invention are preferably suitable for formation of aerosols containing at least two forms of nicotine. Preferable embodiments are powders, liquids, emulsions, and suspensions (e.g., suspensions of microspheres). The formulations may optionally include other drugs, excipients, permeation enhancers, preservatives, absorption enhancers, binding agents, buffers, and the like that enhance the efficacy or ease the use of the claimed invention. Typical nicotine forms of the invention include nicotine dissolved in water or dry powder nicotine with a carrier used to adjust the pH to the desired range. Methods of formulating liquids and liquid inhalers are disclosed in U.S. Pat. Nos. 5,364,838; 5,709,202; 5,497,763; 5,544,646; 5,718,222; 5,660,166; 5,823,178; and 5,910,301; all of which are incorporated by reference to describe and disclose such. Contemplated components of the claimed invention are discussed in greater detail, below.

Powder or granular forms of the invention may be combined with a solution and with a diluting, dispersing or surface-active agent. Additional preferred compositions for administration include a bioadhesive to retain the agent at the site of administration; a spray, paint, or swab applied to the mucosa or epithelium; a slow dissolving pill or lozenge, or the like. The composition may also be in the form of lyophilized powder, which can be converted into solution, suspension, or emulsion before administration. The formulations of the invention are preferably sterilized and stored in unit-dose or multi-dose containers such as sealed vials or ampoules using methods well-known to those of skill in the art.

### A. Suitable Forms of Nicotine

Formulations of the present invention include two forms of nicotine that in combination mimic the pharmacological profile of nicotine delivery produced by a cigarette. The nicotine forms of the invention may be powders, liquids, or encapsulated. Preferably the nicotine forms are suitable for formation of aerosols that are amenable to inhalation. The preparation is such that the inhaled nicotine will be both in a form that provides rapid absorption and also in a form that provides a more sustained rate of absorption. For example, when the claimed formulation is inhaled, the first form of nicotine has a smaller particle diameter than the second form of nicotine. This allows the first form of nicotine to be deposited in the deep lung where it is rapidly transferred to the user's blood stream and reaches the users central nervous system within 5 minutes, preferably in less than 4, 3, 2 or 1 minute. The larger particle size of the second form of nicotine results in deposition of this nicotine form higher up in the respiratory tract. As a result, the second form of nicotine is released more slowly to the users circulatory system with a more sustained effect. Alternatively the treatment is a mixture of immediate and slow release forms of nicotine. Nicotine forms of the invention are discussed in greater detail, below.

1. First Form of Nicotine

The first form of nicotine is preferentially inhaled as this method of administration provides the most rapid delivery without resorting to invasive techniques such as injection. Inhalation allows for a suitable first form of nicotine arterial concentration in the patient within 5 minutes of delivery. Typically this arterial concentration is at least 10, 12, 14 or 15 ng/ml, and this concentration is achieved within 5, preferably within 4, 3, 2, or 1 minute or less from inhalation of the claimed formulation.

To facilitate the rapid delivery of the drug to the user's central nervous system when inhaled, the particle or droplet size of the first form of nicotine is controlled and kept small in order to allow the particles to reach the deep lung. Typically this size is between about 1 μm and about 4 μm in diameter, more preferably about 2 or 3 μm.

The first form of nicotine may have a fluid component having a basic pH, preferably having a pH of more than 7.5, 8.0, or 8.5. A basic pH facilitates formation of the more potent free base form of nicotine. As discussed below, the nicotine forms of the claimed formulation may be encapsulated for example in microspheres. Encapsulation allows the nicotine forms of the formulation to be segregated and therefore they may be delivered with different additives, including buffers adjusting pH, due to their respective microenvironments.

2. Second Form of Nicotine

The second form of nicotine in the formulations of the invention are present in an amount to maintain a second form of nicotine arterial concentration in the patient for at least 60 minutes after delivery. This second form of nicotine formulation, if administered on its own, would lead to an arterial concentration that is generally lower than the first form of nicotine arterial concentration, typically being at least about 8 ng/ml, preferably about 6 ng/ml, more preferably at least about 5 ng/ml, or at least about 4, 3, 2 ng/ml.

Delivery of the second form of nicotine may be performed using any suitable method with preferable methods being buccally (e.g., as a gum, quick dissolve strip, or lozenge composition), transdermal patch, inhalation, or other method that allows for sustained release of the second form of nicotine over a period of several minutes to hours, preferably at least 30, 40, or 60 minutes, more preferably 90 or 120 minutes. The second form of nicotine may be delivered at any pH, but is more preferably delivered at a pH which is most suitable for a particular delivery route.

A preferred method of administering the formulations of the invention is through inhalation. When inhaled, the second form of nicotine may have a larger particle size than the first form of nicotine. As discussed elsewhere in this specification, the larger particle size results in the second form of nicotine being deposited preferentially in the upper respiratory tract rather than the deep lung. Deposition in the higher respiratory airways results in the second form of nicotine reaching the blood system and the receptors of the patient's central nervous system more slowly than is the case for the first form of nicotine deposited in the deep lung. This aids in the sustained release of lower levels of the second form of nicotine to the blood as desired in mimicking the pharmacological administration of nicotine via a cigarette. Thus particles or droplets of the formulation containing the second form of nicotine are preferably in the range between about 4 μm and about 12 μm, more preferably between about 5 μm and about 10 μm, preferentially between about 6 μm and about 8 μm in diameter, as these sizes facilitate deposition of the particles or droplets in the upper airway passages of the lung. It is also possible to slow the absorption using a sustained release formulation.

It is also possible to deliver the second form into the deep lung and other parts of the respiratory tract and provide prolonged elevated levels of nicotine in the arterial blood supply through the sustained release of the second form of nicotine from slow release formulations that are reside over a suitable period of time in the respiratory tract. The residence time may be prolonged by deep lung delivery, or through the use of bioadhesive components. The component of the formulation may optionally include a slow release component such as liposomes or other encapsulating materials well known to those of skill in the art including packaging within microspheres. Encapsulation in microspheres has the added advantage of facilitating delivery of the first and second forms of nicotine at different pH values. For example, the first form of nicotine may be delivered in free base form having a basic pH whereas the second form of nicotine is delivered in salt form as an acidic pH. As is known, the free base form interacts with the nicotinic receptor eliciting a larger response than more acidic? forms of the drug.

Preferred microspheres for use in the invention include polyglycolide microspheres. Microspheres may also optionally include a bioadhesive component such as hyluronic acid.

Microsomes and liposomes of the present invention may be constructed using techniques well-known to those of skill in the art. For example, liposomes containing the second form of nicotine of the present invention may be prepared by suspending a thin layer of purified phospholipids in a solution containing the second form of nicotine and then treating the suspension in a conventional manner such as ultrasonication. A “Liposome” is a closed vesicle of lipid bilayer encapsulating an aqueous compartment therein. It is known that the lipid bilayer membrane structure is extremely similar to biological membranes.

3. Preparing Nicotine Particles

Analysis of Nicotine Containing Aerosols

Purity of a nicotine-containing aerosol may be determined using a number of methods, examples such as described in Sekine et al., Journal of Forensic Science 32:1271 1280 (1987) and Martin et al., Journal of Analytic Toxicology 13:158 162 (1989). One method involves forming the aerosol in a device through which a gas flow (e.g., air flow) is maintained, generally at a rate between 0.4 and 60 L/min. The gas flow carries the aerosol into one or more traps. After isolation from the trap, the aerosol is subjected to an analytical technique, such as gas or liquid chromatography that permits a determination of composition purity.

A variety of different traps are used for aerosol collection. The following list contains examples of such traps: filters; glass wool; impingers; solvent traps, such as dry ice-cooled ethanol, methanol, acetone and dichloromethane traps at various pH values; syringes that sample the aerosol; empty, low-pressure (e.g., vacuum) containers into which the aerosol is drawn; and, empty containers that fully surround and enclose the aerosol generating device. Where a solid such as glass wool is used, it is typically extracted with a solvent such as ethanol. The solvent extract is subjected to analysis rather than the solid (i.e., glass wool) itself. Where a syringe or container is used, the container is similarly extracted with a solvent.

The gas or liquid chromatograph discussed above contains a detection system (i.e., detector). Such detection systems are well known in the art and include, for example, flame ionization, photon absorption and mass spectrometry detectors. An advantage of a mass spectrometry detector is that it can be used to determine the structure of opioid degradation products.

Particle size and composition of the different forms of nicotine of the formulation may be controlled using techniques well-known to those of skill in the art.

Particle size distribution of aerosols produced using formulations of the invention may be determined using any suitable method in the art (e.g., cascade impaction). An Andersen Eight Stage Non-viable Cascade Impactor (Andersen Instruments, Smyrna, Ga.) linked to a source of aerosol by a mock throat (USP throat, Andersen Instruments, Smyrna, Ga.) is one system used for cascade impaction studies.

Inhalable aerosol mass density may be determined, for example, by delivering a drug-containing aerosol into a confined chamber via an inhalation device and measuring the mass collected in the chamber. Typically, the aerosol is drawn into the chamber by having a pressure gradient between the device and the chamber, wherein the chamber is at lower pressure than the device. The volume of the chamber should approximate the tidal volume of an inhaling patient.

Inhalable aerosol nicotine mass density is determined, for example, by delivering an aerosol of the invention into a confined chamber via an inhalation device and measuring the amount of active drug compound collected in the chamber. Typically, the aerosol is drawn into the chamber by having a pressure gradient between the device and the chamber, wherein the chamber is at lower pressure than the device. The volume of the chamber should approximate the tidal volume of an inhaling patient. The amount of nicotine collected in the chamber is determined by extracting the chamber, conducting chromatographic analysis of the extract and comparing the results of the chromatographic analysis to those of a standard containing known amounts of drug.

Inhalable aerosol particle density is determined, for example, by delivering an aerosol of the invention into a confined chamber via an inhalation device and measuring the number of particles of given size collected in the chamber. The number of particles of a given size may be directly measured based on the light-scattering properties of the particles. Alternatively, the number of particles of a given size is determined by measuring the mass of particles within the given size range and calculating the number of particles based on the mass as follows: Total number of particles=Sum (from size range 1 to size range N) of number of particles in each size range. Number of particles in a given size range=Mass in the size range/Mass of a typical particle in the size range. Mass of a typical particle in a given size range=π*D³*θ/6, where D is a typical particle diameter in the size range (generally, the mean boundary MMADs defining the size range) in microns, θ is the particle density (in g/mL) and mass is given in units of picograms (g⁻¹²).

Rate of inhalable aerosol particle formation is determined, for example, by delivering aerosol into a confined chamber via an inhalation device. The delivery is for a set period of time (e.g., 3 s), and the number of particles of a given size collected in the chamber is determined as outlined above. The rate of particle formation is equal to the number of 100 nm to 5 μm particles collected divided by the duration of the collection time.

Rate of aerosol formation is determined, for example, by delivering aerosol phase drug into a confined chamber via an inhalation device. The delivery is for a set period of time (e.g., 3 s), and the mass of particulate matter collected is determined by weighing the confined chamber before and after the delivery of the particulate matter. The rate of aerosol formation is equal to the increase in mass in the chamber divided by the duration of the collection time. Alternatively, where a change in mass of the delivery device or component thereof can only occur through release of the aerosol phase particulate matter, the mass of particulate matter may be equated with the mass lost from the device or component during the delivery of the aerosol. In this case, the rate of aerosol formation is equal to the decrease in mass of the device or component during the delivery event divided by the duration of the delivery event.

Dry Powder Formulations

Methods for producing dry powder formulations with particle sizes limited to an inhalable aerodynamic are well known by those of skill in the art. They include but are not limited to, milling, spray-drying, freeze-drying, lyophilization, absorption and adsorption of active ingredients into and onto carrier particles.

Dry powder formulations obtainable according to the invention may include a pharmaceutically inactive carrier of noninhalable particle size, a finely divided pharmaceutically active compound of inhalable particle size and to improve the resistance to moisture—magnesium stearate, and they are preferably present in the form of “interactive (or ordered or adhesive) mixtures”. If desired, the dry powder formulations can also contain a proportion of carrier material of inhalable particle size. In principle, the constituents can be mixed with one another in any desired sequence, where, however, mixing should expediently be carried out in such a way that the particles of the constituents are essentially retained as such, i.e. are not destroyed, for example, by granulation and the like. Mixing can be carried out in a manner known per se, for example in a tumble mixer.

The expression “interactive mixture” or “ordered mixture” or “adhesive mixture” is familiar to the person skilled in the art and in the context of the present invention comprises dry powder formulations in which the pharmacologically inactive carrier is present in a particle size which is noninhalable or mainly noninhalable, and in which microfine particles of the nicotine forms are bound to the carrier particles by adhesion (i.e. are not contained in the carrier, e.g. in the form of granules).

The amount of nicotine in the formulations obtainable according to the invention may vary within wide ranges and is to a high extent dependent on the particular nicotine form and up to a certain degree also on the powder inhaler used. Typically, the nicotine concentration can be approximately 0.1 to 10% by weight, in particular approximately 0.1 to 5% by weight, based on the total formulation. Occasionally, higher or lower concentrations can also be expedient however active compound concentrations of below 0.001% by weight or below 0.01% by weight rarely occur.

Microsphere Formulations

Preparation of Microspheres, Including Liposomes is Well Known in the Art, as are compositions providing microspheres with different dissolution rates. Thus formulations of the invention may include lipophilic substances that can enhance absorption of the agent through the mucosa or epithelium of the nasal cavity. The forms of nicotine of the invention may be mixed with a lipophilic adjuvant alone or in combination with a carrier, or may be combined with one or several types of micelle or liposome substances. Among the preferred lipophilic substances are cationic liposomes included of one or more of the following: phosphatidyl choline, lipofectin, DOTAP, a lipid-peptoid conjugate, a synthetic phospholipid such as phosphatidyl lysine, or the like. These liposomes may include other lipophilic substances such as gangliosides and phosphatidylserine (PS). Also preferred are micellar additives such as GM-1 gangliosides and phosphatidylserine (PS), which may be combined with the agent either alone or in combination. GM-1 ganglioside can be included at 1-10 mole percent in any liposomal compositions or in higher amounts in micellar structures. Protein agents can be either encapsulated in particulate structures or incorporated as part of the hydrophobic portion of the structure depending on the hydrophobicity of the active agent.

For one skilled in the art, the release rate from microspheres can be easily modified ranging from days to months by altering the ratio of the copolymers. For example, the Eligard product uses the ATRIGEL® Delivery System, a polymeric (non-gelatin containing) delivery system consisting of a biodegradable poly(DL-lactide-co-glycolide) (PLGH) polymer formulation dissolved in a biocompatible solvent, N-methyl-2-pyrrolidone (NMP). The leuprolide delivery rate is described by a one-month release by using co-polymer with a 50:50 molar ratio of DL-lactide to glycolide containing carboxyl end groups. In the 3 and 6-month product, the leuprolide delivery rate is achieved by using co-polymer with a 75:25 molar ratio of DL-lactide to glycolide with hexanediol or an 85:15 molar ratio of DL-lactide to glycolide with hexanediol, respectively. Clearly, the greater the ratio of PLA to PGA the more prolonged the release.

Examples of commercially available peptide/protein controlled, release systems based on PLGA include:

| Drug | Trade Name | Company | Polymer | Route | Application |
|---|---|---|---|---|---|
| buserelin | Profact□Depot, | Hoechst | PLGA | s/c implant | Prostate |
| acetate | Suprefact□Depot | Marion | | | cancer |
| | | Roussel | | | |
| goserelin | Zoladex□Depot | Astra Zeneca | PLGA | s/c implant | Prostate |
| acetate | | | | | cancer, |
| | | | | | endometrioses |
| Leuprolide | Eligard | Sanofi- | | 1, 3, 4 and 6 | Prostate |
| acetate | | aventis | | month | cancer |
| | | | | suspension | |
| | | | | injection | |
| leuprorelin | Lupron□Depot, | Takeda- | PLGA | 3-month | Prostate |
| acetate | Enantone□Depot, | Abbott | PLA | depot | cancer, |
| | Enantonen□Gyn | | | suspension, | endometrioses |
| | Depot | | | 1-month | |
| | Trenantone□ | | | suspension | |
| | | | | 3-month | |
| | | | | suspension | |
| octreotide | Sandostatin | Novartis | PLGA | s/c | GH |
| acetate | LAR□Depot | Pharma | | suspension | suppression, |
| | | | | | anti cancer |
| triptorelin | Decapeptyl ® | Debiopharma | PLGA | s/c depot | LHRH agonist, |
| | Depot | | | injection | prostate |
| | | | | | cancer |
| recombinant | Nutropin□Depot, | Genentech- | PLGA | monthly s/c | Growth |
| human | [discontinued | Alkermes | | injection | hormone |
| growth | commercialisation | | | | deficiency |
| hormone | since June 2004] | | | | |

One preferred liposomal formulation employs Depofoam. An agent can be encapsulated in multivesicular liposomes, as disclosed in the copending application entitled “High and Low Load Formulations of IGF-I in Multivesicular Liposomes,” U.S. patent application Ser. No. 08/925,531, filed Sep. 8, 1997, herein incorporated by reference. The mean residence time of agent at the site of administration can be prolonged with a Depofoam composition.

4. Supplemental Drugs

Methods for formulating pharmaceutical compositions are generally known in the art. A thorough discussion of formulation and selection of pharmaceutically acceptable carriers, stabilizers, and isomolytes can be found in Remington's Pharmaceutical Sciences (18.sup.th ed.; Mack Publishing Company, Eaton, Pa., 1990), herein incorporated by reference.

In addition to the nicotine forms discussed above, the tobacco-less compositions of the present invention may optionally include supplemental pharmaceutically-active components. These supplemental components may aid in delivery of the nicotine forms of the formulation, provide further support of the patient's program to terminate their nicotine addiction, treat diseases, or make the formulations of the invention more acceptable to the patient-user.

Particularly preferred supplemental drugs include antidepressants and anxiolytics such as selective serotonin reuptake inhibitors, e.g., citalopram, escitalopram, fluoxetine, paroxetine, sertraline, and the like. Serotonin and norepinephrine reuptake inhibitors are also preferred, such as duloxetine, venlafaxine, and the like. Norepinephrine and dopamine reuptake inhibitors such as bupropion may also be used. Tetracyclic antidepressants such as mirtazapine; combined reuptake inhibitors and receptor blockers such as trazodone, nefazodone, maprotiline; tricyclic antidepressants, such as amitriptyline, amoxapine, desipramine, doxepin, imipramine, nortriptyline, protriptyline and trimipramine; monoamine oxidase inhibitors, such as phenelzine, tranylcypromine, isocarboxazid, selegiline; benzodiazepines such as lorazepam, clonazepam, alprazolam, and diazepam; serotonin 1A receptor agonists such as buspirone, aripiprazole, quetiapine, tandospirone and bifeprunox; and a beta-adrenergic receptor blocker, such as propranolol may also be added to enhance the claimed tobacco-less formulations of the present invention.

The formulations of the present invention may also optionally include other pharmacologic agents such as UTP, amiloride, antibiotics, bronchodilators, anti-inflammatory agents, and mucolytics (e.g. n-acetyl-cysteine). In addition to including other therapeutic agents in the formulation itself, the formulations of the present invention may also be administered sequentially or concurrently with the one or more other pharmacologic agents identified herein. The amounts of formulation and pharmacologic agent depend, for example, on what type of pharmacologic agent(s) are used, and the scheduling and routes of administration

Supplemental drugs may be delivered concomitantly with the formulations of the present invention, or may be administered independently. Supplemental drug delivery may be via any suitable method known in the art including orally, inhalation, injection, etc.

### B. Pharmaceutically Acceptable Excipients

The formulations of the present invention are administered to a human and may contain one or more pharmaceutically-acceptable excipients, or carriers. Suitable excipients and their formulations are described in Remington's Pharmaceutical Sciences, 16th ed., 1980, Mack Publishing Co., edited by Oslo et al.

For the exact volumetric dosage of the formulations of the invention, dilution of the active compound with a pharmaceutically inactive excipient may be necessary in order to obtain a dosable unit amount meeting the demands on dosage accuracy. Where necessary the dilution is chosen such that the amount applied from the inhaler exactly contains the desired dose. The pharmacologically inactive excipient preferably serves not only for dilution, but also for the adjustment of a flowability of the powder mixture or aerosol mist. The proportion of carrier material in the formulations obtainable according to the invention can vary within a wide range depending on the dilution. The proportion of carrier material to the total formulation can be, for example, approximately 80 to 99.9% by weight, where, however, higher or lower proportions can also be advantageous depending on the nicotine form(s) of the formulation. The lower proportion of the excipient is advantageous in order to minimize the possibility of adverse reactions due to the excipient, unless the excipient is known to be safe when delivered by inhalation.

The carrier is preferably present in the formulation obtainable according to the invention in a particle size which is not inhalable. The carrier particles, however, should on the other hand not be too large, as this can have a disadvantageous effect on the FPF. The optimum particle size of the carrier employed in this case as a rule depends on the demands and specifications of the inhaler which is intended for the administration of the formulation. In the context of the present invention, carriers having customary particle sizes can be used, and optimum particle sizes can easily be determined from case to case by the person skilled in the art. In general, however, the mean particle diameter (MMAD) of the carrier particles can be approximately 10 to 500 μm and preferably approximately 50 to 200 μm.

Where applicable, the adhesion of the formulation particles to carrier particles should be sufficient that no demixing takes place during processing, transport, storage and dosage operations, but on the other hand not so high that a detachment of the formulation particles which is as quantitative as possible is no longer guaranteed during the dispersion in the inhaler induced by the respiratory flow of the patient. The effectiveness of the release of the active compound particles is especially dependent, in addition to the physicochemical properties of the active compound and the aerodynamic properties of the powder inhaler, on the properties of the carrier, in particular the nature of the carrier and its surface structure, mean particle size and particle size distribution.

In the context of the powder formulations of the present invention, fundamentally all carrier materials customarily used in dry powder formulations are suitable, for example mono- or disaccharides, such as glucose, lactose, lactose monohydrate, sucrose or trehalose, sugar alcohols, such as mannitol or xylitol, polylactic acid or cyclodextrin, glucose, trehalose and in particular lactose monohydrate in general being preferred. If desired, the formulations can also contain two or more carrier materials. If desired, in addition to noninhalable carrier particles, the formulation can also contain a proportion of inhalable carrier particles; for example in addition to relatively coarse lactose monohydrate carrier particles it can contain a proportion of, for example, 0.1 to 10% by weight of micronized lactose monohydrate, which can have, for example, a particle size diameter of at most 10 μm, preferably at most 5 μm, for at least 50% of the particles.

Water, saline, aqueous dextrose, and glycols are preferred liquid carriers, particularly (when isotonic) for solutions. The carrier can be selected from various oils, including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. The compositions can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like.

Other optional components of the formulations may include, but are not limited to, buffers that enhance isotonicity such as water, saline, phosphate, citrate, succinate, acetic acid, and other organic acids or their salts. Typically, the pharmaceutically acceptable carrier also includes one or more stabilizers, reducing agents, anti-oxidants and/or anti-oxidant chelating agents. The use of buffers, stabilizers, reducing agents, anti-oxidants and chelating agents in the preparation of protein based compositions, particularly pharmaceutical compositions, is well-known in the art. See, Wang et al., “Review of Excipients and pHs for Parenteral Products Used in the United States.” J. Parent. Drug Assn., 34(6):452-462 (1980); Wang et al., “Parenteral Formulations of Proteins and Peptides: Stability and Stabilizers,” J. Parent. Sci. and Tech., 42:S4-S26 (Supplement 1988); Lachman, et al., “Antioxidants and Chelating Agents as Stabilizers in Liquid Dosage Forms-Part 1,” Drug and Cosmetic Industry, 102(1): 36-38, 40 and 146-148 (1968); Akers, M. J., “Antioxidants in Pharmaceutical Products,” J. Parent. Sci. and Tech., 36(5):222-228 (1988); and Methods in Enzymology, Vol. XXV, Colowick and Kaplan eds., “Reduction of Disulfide Bonds in Proteins with Dithiothreitol,” by Konigsberg, pages 185-188.

Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. See Wang (1980) at page 455. Suitable salts and isotonicifiers include sodium chloride, dextrose, mannitol, sucrose, trehalose, or the like. Where the carrier is a liquid, it is preferred that the carrier is hypotonic or isotonic with oral, conjunctival or dermal fluids and have a pH within the range of 4.5-8.5. Where the carrier is in powdered form, it is preferred that the carrier is also within an acceptable non-toxic pH range.

The formulations may also include an adjuvant such as cetyl trimethyl ammonium bromide, BDSA, cholate, deoxycholate, polysorbate 20 and 80, fusidic acid, or the like, and in the case of DNA delivery, preferably, a cationic lipid. Suitable sugars include glycerol, threose, glucose, galactose and mannitol, sorbitol. A suitable protein is human serum albumin.

Preferred fluid compositions include one or more of a solubility enhancing additive, preferably a cyclodextrin; a hydrophilic additive, preferably a mono or oligosaccharide; an absorption promoting additives, preferably a cholate, a deoxycholate, a fusidic acid, or a chitosan; a cationic surfactant, preferably a cetyl trimethyl ammonium bromide; a viscosity enhancing additive, preferably to promote residence time of the composition at the site of administration, preferably a carboxymethyl cellulose, a maltodextrin, an alginic acid, a hyaluronic acid, or a chondroitin sulfate; or a sustained release matrix, preferably a polyanhydride, a polyorthoester, a hydrogel, a particulate slow release depo system, preferably a polylactide co-glycolides (PLG), a depo foam, a starch microsphere, or a cellulose derived buccal system; a lipid based carrier, preferably an emulsion, a liposome, a niosomes, or a micelles. The composition can include a bilayer destabilizing additive, preferably a phosphatidyl ethanolamine; a fusogenic additive, preferably a cholesterol hemisuccinate.

Pharmaceutically acceptable excipients may be volatile or nonvolatile. Volatile excipients, when heated, are concurrently volatilized, aerosolized and inhaled with the antihistamine. Classes of such excipients are known in the art and include, without limitation, gaseous, supercritical fluid, liquid and solid solvents. The following is a list of exemplary carriers within the classes: water; terpenes, such as menthol; alcohols, such as ethanol, propylene glycol, glycerol and other similar alcohols; dimethylformamide; dimethylacetamide; wax; supercritical carbon dioxide; dry ice; and mixtures thereof.

These lists of carriers and additives is by no means complete and a worker skilled in the art can choose excipients from the GRAS (generally regarded as safe) list of chemicals allowed in the pharmaceutical preparations and those that are currently allowed in topical and parenteral formulations.

### C. Propellants

Tobacco-less formulations of the present invention may also include a propellant suitable for aerosolizing the pharmaceutically active nicotine formulation. Suitable propellants are well-known in the art and include compressed air, nitrogen, hydrofluoroalkanes (HFAs) and the like. An important aspect of any propellant used in the present invention is that it not react with nicotine or other pharmaceutically-active components of the tobacco-less formulations of the claimed invention.

### III. Methodology

The penetration of aerosolized nicotine particles into the respiratory tract is determined largely by the size distribution of the particles formed and may be also affected by the breathing pattern just prior to, during and just after the inhalation of the medication. The sites of deposition also depend on age and the pathophysiological condition of the person inhaling the medication. Under normal breathing conditions, larger particles, i.e., particles with a diameter greater than or equal to 5 μm, deposit predominantly on the upper airways of the lungs (see FIG. 1). Particles having a diameter in a range of about >2 microns (μm) to <5 microns (μm) deposit predominantly in the central airways. Smaller particles having a diameter □2 microns (μm) penetrate predominantly into the peripheral region of the lungs.

In one aspect of the invention the treatment methodology begins with particles of a given size, carries out treatment for a given period of time after which the particles are increased in size. The particles initially administered to the patient penetrate deeply into the lung, i.e., the smallest particles (e.g., 0.5 to 2 microns (μm)) target the alveolar ducts and the alveoli. When the deepest part of the lung is targeted with the smallest particles the patient receives an immediate “rush” from the nicotine delivered which closely matches that received when smoking a cigarette. These small particles can be obtained by any method that produces inhalable particles, such as by milling powder into the desired size and inhaling the powder or by creating a solution or suspension and aerosolizing the formulation, e.g. by nebulization or by moving the solution or suspension through the pores of a membrane. In either case, the desired result is to obtain particles which have a diameter in the range of 0.5 μm to about 2 μm. Those skilled in the art will understand that some of the particles will fall above and below the desired range. However, if the majority of the particles (50% or more) fall within the desired range then the desired area of the lung will be correctly targeted.

In practicing the present invention, the patient is allowed to repeatedly administer the tobacco-less formulation of the invention when a cigarette is desired. For example, the patient would be instructed to repeatedly administer the tobacco-less formulation when the patient would normally smoke a cigarette. In this manner, the patient will become accustomed to finding that the device administers nicotine into the patient in the same manner that a cigarette does. In one embodiment of the invention the concentration of the nicotine in the tobacco-less formulation could be reduced gradually over time. This could be done over a sufficiently long period of time so as to allow the patient to wean off of nicotine. However, in another embodiment of the invention the amount of nicotine is kept substantially constant but the size of the aerosolized particles created are increased.

In another treatment methodology, the patient would begin the treatment with a low dose of the tobacco-less formulation of the invention and this dosage would gradually be raised as the patient grew more tolerant of the formulation. With the increasing tobacco-less formulation dosage, the patient could gradually cease smoking until the tobacco-less formulation completely replaced the cigarette. Administration of a constant dose of antidepressant or anxiolytic throughout this process may further improve the patient's probability of overall success. Once the cigarette habit is broken, the patient would gradually lower the dosage of the tobacco-less formulation until the nicotine addiction was broken. The continued use of the antidepressant or anxiolytic could enhance the patient's ability to wean themselves off the tobacco-less nicotine formulation.

Another treatment methodology would gradually increase the size of the particles for the first form of nicotine. The increased particle size targets predominantly the respiratory tract above the alveolar ducts and below the small bronchi. This can generally be accomplished by creating aerosolized particles of nicotine which have a size and range of about 2 μm to about 4 μm. Administration is carried out in the same manner as described above. Specifically, the patient administers the aerosolized nicotine at the same time when the patient would be smoking a cigarette. Since the patient has become adjusted to receiving the nicotine “rush” from the smaller sized particles, the patient will expect and is therefore likely to experience the same “rush” when administering the slightly larger particles. However, the effect will be less immediate as a consequence of the particles being deposited predominantly in a higher region of the respiratory tract. This procedure is carried out over a period of time, e.g., days or weeks. In one embodiment of the invention it is possible to reduce the dose of aerosolized nicotine delivered to the patient during this second phase. However, the dose may remain constant.

The treatment can be completed after any phase, e.g. after the second phase. However, in accordance with a more preferred embodiment of the invention a third phase of treatment is carried out. Within the third phase the particle size of the first form of nicotine is increased again. The particles are increased to a size in a range from about 4 μm to about 8 μm or, alternatively, perhaps as large as 12 μm. These larger particles will target predominantly the upper airways. The larger particles will give a very small immediate “rush” but will still be absorbed through the mucous membranes of the patient's respiratory tract. Accordingly, the patient will be administering nicotine doses which may be the same as those doses administered at the beginning of treatment. At this point the treatment can take a number of different directions. The patient can attempt to stop administration by immediate and complete cessation of nicotine delivery. Alternatively, the patient can try to wean off of nicotine by delivering fewer doses during a given time period, or by decreasing the dose per use, as discussed below.

In another alternative, the same size dose (volume of aerosol formulation) is administered and delivered, creating the same amount of aerosol, but wherein the aerosolized particles contain progressively less nicotine (e.g., more dilute concentration of nicotine in the particles or droplets). The amount of nicotine can be decreased until the patient is receiving little or no nicotine. Those skilled in the art reading this disclosure will recognize variations on the overall method and methods for stopping treatment.

In yet another alternative embodiment the amount of nicotine, concentration of nicotine and particle sizes created by the formulation are all maintained the same from one group of packets to the next. However, the pH of the formulation within the packets from one group to the next is changed and is generally changed from a high or basic pH to a low or acidic pH. Thus, for example, the pH of the packets within a first group could be at 9.0 and the pH of the formulation in a second group of packets could be 8.0, followed by a third group at 7.0 followed by a fourth group at 6.0 followed by a fifth group at 5.0. Those skilled in the art, reading this disclosure will understand that the variation in pH from one group to the next can be in any amount and the pH can begin and end at any point provided the resulting formulation does not cause damage to the lungs of the patient to an unacceptable degree. In preferred embodiments, the pH of the first form of nicotine is varied from basic to acid thereby gradually decreasing the amount of free base nicotine in the formulation. The pH of the second form of nicotine may also be adjusted, but preferably remains constant, typically at a neutral or acidic pH level.

In yet another embodiment of the invention the nicotine forms of the invention may include variations of all or any of the different parameters which include amount of nicotine, concentration of nicotine, particle size of aerosol created and pH of the formulation. Any one, two, three or four of the parameters can be varied from one administration to the next.

Supplemental Treatment Methodology

Tobacco users wishing to quit may be treated solely with respiratory nicotine as indicated above, i.e. by intrapulmonary delivery. However, it is possible to treat such patients with a combination of pulmonary administration and other means of administration, such as transdermal administration. Transdermal nicotine is preferably administered to maintain a steady state level of nicotine within the circulatory system. Nasal or buccal formulation could be used for nasal or buccal delivery which could supplement aerosolized delivery.

Supplemental nicotine treatments may be combined with administration of the formulations of the present invention in an effort to augment nicotine treatment. As noted above, the exemplary supplemental treatments identified below typically do not provide the rapid increase in arterial nicotine concentration provided by the formulation of the present invention. The supplements do however generally provide a basal sustained nicotine level that contributes to the effect of the second forms of nicotine of the present invention. The supplemental nicotine treatments listed below are exemplary only and do not constitute an exhaustive list.

By way of example, transdermal nicotine delivery systems include nicotine patches and others that are described in the art for example, in U.S. Pat. Nos. 4,597,961, 5,004,610, 4,946,853, and 4,920,989, each of which is expressly incorporated herein by reference.

Transmucosal administration is also known, for example delivery of nicotine to the systemic circulation through oral drug dosage forms (e.g., lozenge, capsule, gum, tablet, suppository, ointment, gel, pessary, membrane, and powder) are typically held in contact with the mucosal membrane and disintegrate and/or dissolve rapidly to allow immediate systemic absorption. This term includes, but is not limited to, lozenges, capsules, tablets, and gum. These formulations can also be such that they provide slow, sustained release of nicotine yielding prolonged arterial blood concentrations of nicotine.

Preferably, the orally administrable nicotine formulation will consist of any lozenge, tablet, capsule, or gum formulation that delivers nicotine through the oral mucosa cavity, and preferably through the buccal and/or sublingual mucosa. The nicotine form that is added or incorporated into the nicotine formulations may be pure nicotine or any compound thereof. The method of manufacture of these formulations may be any suitable method known in the art, including but not limited to the addition of a nicotine compound to premanufactured tablets; cold compression of an inert filler, a binder, and either pure nicotine or a nicotine-containing substance (as described in U.S. Pat. No. 4,806,356, herein incorporated by reference); encapsulation of nicotine or a nicotine compound; and incorporation of nicotine bound to a cation exchange resin, for example, as in a chewing gum (as described in U.S. Pat. Nos. 3,877,468 and 3,901,248, herein incorporated by reference).

Based on the above, it will be understood by those skilled in the art that a plurality of different treatments and means of administration can be used to treat a single patient. For example, a patient can be simultaneously treated with nicotine by transdermal administration, nicotine via pulmonary administration, in accordance with the present invention, and nicotine which is administered to the mucosa.

### IV. Nicotine Delivery Devices

The aspects of the invention described above such as changing the amount, concentration, or pH of the formulation or changing the particle size of the aerosol created with the formulation can be done independent of the delivery device. However, there are a number of features which can be included in the system which are specific to the device which delivers the formulation. For example, the device can be designed so as to avoid overdosing. This can be carried out by electronically monitoring the number of doses a patient has delivered and locking out further use for a given time interval. Thus, this system can be used as a safety feature. In addition to a safety feature the device can be programmed in order to force the frequency of administration. This could be done in order to aid the patient in reducing the times the dose is delivered and thereby moving the patient forward towards a point in time when the patient no longer needs nicotine.

Devices, if desired, contain a variety of components to facilitate the delivery of the formulations of the invention. For instance, the device may include any component known in the art to control the timing of drug aerosolization relative to inhalation (e.g., breath-actuation), to provide feedback to patients on the rate and/or volume of inhalation, to prevent excessive use (i.e., “lock-out” feature), to prevent use by unauthorized individuals, and/or to record dosing histories.

Any of the devices suitable for use with the invention could be designed to force the patient to use only a certain dosage form of the tobacco-less formulation for a given period of time and then require that the patient use another dosage form. In this way the device can be programmed to start the patient with, for example, a relatively high dose which can be quickly administered and thereafter allowing the device only to be activated when a second group with a smaller amount, lower concentration, etc. is used in the device.

The devices suitable for use with the invention can also be programmed to be patient and physician specific. Thus, the device can include a lock-out component which prevents the device being used except in the presence of another component which could, for example, be a wristband worn by the patient. The device could also be programmable only by a particular physician equipped with a device which sends a signal allowing the device to be reprogrammed.

Devices suitable for use with the invention can also be programmed to release larger or lesser amounts of formulation and fire the aerosol at different rates. Either or both of these parameters can be changed by themselves, together or in combination with the other parameters relating to the formulation and particle size.

Although any device suitable for delivering the requisite amounts of formulation to the lungs of a patient may be employed to deliver the formulations of the invention, the Aradigm AERx Essence® is preferred.

Precision delivery of small molecule drugs via the lung for systemic effect is possible. An electronic inhaler capable of delivering a liquid formulated drug stored in a unit dose packages has been described and disclosed in U.S. Pat. No. 5,718,222 entitled “Disposable Package for Use in Aerosolized Delivery of Drugs,” and is incorporated herein by reference. A formulation of nicotine can be prepared for delivery with this system. Quantitative delivery of nicotine on demand provides a mechanism for nicotine replacement therapy which is unlikely to be associated with recidivism precipitated by the symptoms of physical withdrawal.

In one embodiment, the tobacco-less nicotine formulation of the invention is forced through the openings or pores of a porous membrane to create an aerosol. In a specific embodiment, the openings are all uniform in size and are positioned at uniform distances from each other. However, the openings can be varied in size and randomly placed on the membrane. If the size of the openings is varied, the size of the particles formed will also vary. In general, it is preferable to have the opening sizes within the range of about 0.25 μm to about 6 μm which will create particle sizes of about 0.5 μm to 12 μm which are preferred with respect to inhalation applications. When the openings have a pore size in the range of 0.25 μm to 1 μm they will produce an aerosol having particle sizes in the range of 0.5 μm to 2 μm, which is particularly useful for delivering nicotine to the alveolar ducts and alveoli. Pore sizes having a diameter of about 1 μm to 2 μm will produce particles having a diameter of about 2 μm to 4 μm, which are particularly useful for delivering nicotine to the area above the alveolar ducts and below the small bronchi. A pore size of 2 μm to 4 μm will create particles having a diameter of 4 μm to 8 μm, which will target the area of the respiratory tract from the small bronchi upward.

Increasing the size of the openings of the porous membranes produces nicotine formulation particles of increasing size. A strategy in which the blood levels of nicotine, and especially the peak levels, are reduced gradually will be the most effective in treating the symptoms of withdrawal, and thereby increase the chances of successful smoking cessation. In one embodiment of the invention, the size of the aerosolized nicotine particles is increased in a stepwise manner by using porous membranes that create “monodisperse” aerosols, wherein all the particles within the aerosol created have essentially the same particle size. Nicotine particles of increasing size are produced by using membranes of increasing pore sizes.

In another embodiment, the size of aerosolized tobacco-less nicotine formulation particles is increased in gradient fashion by using porous membranes that create “multi-disperse” aerosols, wherein the particles within the aerosol created have different particle sizes. Membranes which have an increasing range of pore sizes are used to produce nicotine particles of increasing size.

As intrapulmonary administration is not 100% efficient, the amount of drug aerosolized will be greater than the amount that actually reaches the patient's circulation. For example, if the inhalation system used is only 50% efficient then the patient will aerosolize a dose which is twice that needed to raise the patient's nicotine level to the extent needed to obtain the desired results. More specifically, when attempting to administer 1 mg of nicotine with a delivery system known to be 50% efficient, the patient will aerosolize an amount of formulation containing about 2 mg of nicotine.

A device comprised of a container that includes an opening covered by a porous membrane, such as the device disclosed in U.S. Pat. No. 5,906,202, may be used to deliver nicotine. The device may be designed to have the shape and/or bear the markings of a pack of cigarettes, and may include the scent of tobacco. These features and others that address the behavioral component of cigarette smoking may enhance the effectiveness of the method described herein.

Containers for the formulations of the present invention may be any form suitable for use with the chosen delivery system. Preferred containers for formulations designed to be delivered as aerosols are single dose packets, for example blister packets containing a liquid sterile formulation of the invention. In one embodiment, the volume of the receptacle is at least about 0.037 cm³. In another embodiment, the volume of the receptacle is at least about 0.048 cm³. In yet another embodiment, are receptacles having a volume of at least about 0.067 cm³ or 0.095 cm³. In one embodiment of the invention, the receptacle is a capsule that holds try powder containing nicotine designated with a capsule size 2, 1, 0, 00 or 000. Suitable capsules can be obtained, for example, from Shionogi (Rockville, Md.). Blisters designed to hold powder formulations can be obtained, for example, from Hueck Foils, (Wall, N.J.).

### V. Dosing

A tobacco cigarette contains 6 to 11 mg of nicotine, of which the smoker typically absorbs 1 to 3 mg; see Henningfield N Engl J Med 333:1196-1203 (1995). Factors influencing nicotine absorption include subject-dependent factors, such as smoking behavior, lung clearance rate, etc., morphological factors, and physiological factors, such as tidal volume, inspiratory and expiratory flow rate, particle size and density. See Darby et al., Clin Pharmacokinet 9:435-439 (1984). The systemic dose of nicotine per puff is extremely variable, however, peak plasma concentrations of 25 to 40 ng/mL of nicotine, achieved within 5 to 7 minutes by cigarette smoking, are believed typical. In accordance with the present invention, about 0.05 to about 3 mg, preferably about 0.3 to about 1 mg, preferentially about 0.3 to about 0.7 mg of nicotine are delivered to the lungs of the patient in a single dose to achieve peak blood plasma concentrations of 10 to 40 ng/mL. These specific amounts should not be relied on. Alternatively, the amounts should be measured, adjusted, remeasured and readjusted as needed to obtain the appropriate dosing. An aspect of the invention is to initially set out to deliver the nicotine preparation in a manner that satisfies the craving for high plasma levels of nicotine in the subject and then gradually changing the nature of the inhaled nicotine formulation in terms of the amount of nicotine, its concentration as well as site of deposition so as to gradually reduce the peak plasma nicotine levels to wean the subject off tobacco smoking. The amount needed will vary based on many factors including how much the patient smokes, and the patient's age, sex, weight and condition.

The amount of nicotine administered will vary based on factors such as the age, weight and frequency of smoking or nicotine tolerance of the smoker. Other factors, such as daily stress patterns, and demographic factors may also help to determine the amount of nicotine sufficient to satisfy the smoker's craving for the drug. Administering nicotine using the methods of the present invention can involve the daily administration of anywhere from 0.05 mg to 200 mg of nicotine, but more preferably involves the administration of approximately 1 to 100 mg per day, but these amount ranges should not be relied on. Amounts should be determined as indicated above.

When nicotine enters the circulatory system of a human patient it is oxidized to cotinine within four to six hours. The present invention includes the administration of cotinine and other nicotine derivatives provided such derivatives do not result in unacceptable adverse effects.

Methods of Administering Formulations

The tobacco-less formulations described herein may be administered by systemic injection, transdermal administration by applying the medicament directly to the skin, oral ingestion, inhalation as described herein, or by other methods such as systemic infusion. Commercially available nebulizers for liquid formulations, including jet nebulizers and ultrasonic nebulizers may be useful for administration. Liquid formulations may be directly nebulized and lyophilized power nebulized after reconstitution. Alternatively the tobacco-less formulation may be aerosolized using a metered dose inhaler, or inhaled as a powder that could be prepared by any of the methods known to those skilled in the art. For example, the powder could be prepared by lyophilization, spray-drying, freeze-drying, milling, or by incorporation of nicotine into premanufactured particles or entrapment in microparticles. In addition, a liquid medicament may be directly instilled in the nasotracheal or endotracheal tubes in intubated patients.

Effective dosages and schedules for administering the formulations may be determined empirically, and making such determinations is within the skill in the art Those skilled in the art will understand that the dosage of tobacco-less formulation of the invention that must be administered will vary depending on, for example, the person receiving the formulation, the route of administration, the particular type of formulation used and other drugs being administered to the patient. As previously noted, the formulation of the present invention may be administered in a single dose, or as multiple doses over time.

The formulations are typically administered in a dose sufficient to provide a therapeutically effective level. By way of example, nicotine arterial concentration produced by the first nicotine form is preferably at least 10 ng/ml, but may be 15, 20, 25, 30, 35, 40, 50 or more ng/ml nicotine, being limited by the amount necessary to address the nicotine addiction while not reaching toxic levels. Similarly, the second form of nicotine is administered in an amount to maintain a second form of nicotine arterial concentration in the patient for at least 60 minutes after administration. This may be augmented by addition of slow release components such as cyclodextrin, encapsulation of the active nicotine, chemical or physical modification of the form of nicotine and the like as described herein. The maintained arterial concentration of the second form of nicotine is at least 5 ng/ml, preferably 7, 10, 12, 15 or 20 ng/ml.

It would be apparent to a person skilled in the art that variations may be acceptable with respect to the therapeutically effective dose and frequency of the administration of formulations of the invention. For example, the amount of the formulation administered may be inversely correlated with the frequency of administration. For example, an increase in the concentration of neurologic agent in a single administered dose, or an increase in the mean residence time in the case of a sustained release form of neurologic agent, generally will be coupled with a decrease in the frequency of administration.

It is appreciated by those of skill in the art that the actual formulation dose will depend on a variety of factors that may be specific to the subject undergoing dosing. These factors should be taken into consideration when determining the therapeutically effective formulation dose and frequency of its administration. For example, the effective dose can depend on the age, weight, or general health of the subject; the severity of the nicotine addiction; the frequency and duration of dosing; the type of formulation administered; the characteristics, such as lipophilicity, of the formulation and composition; and the like. Generally, a higher dosage is preferred if the nicotine addiction is more severe. Thus some minor degree of experimentation may be required to determine the most effective dose and frequency of dose administration, this being well within the capability of one skilled in the art once apprised of the present disclosure.

Intermittent Dosing

In another embodiment of the invention, the therapeutically effective formulation is administered intermittently. “Intermittent administration” is intended administration of a therapeutically effective formulation dose followed by a time period of discontinuance, which is then followed by another administration of a therapeutically effective dose, and so forth. “Time period of discontinuance” is intended a discontinuing of daily administration of the formulation. During the time period of discontinuance, the arterial nicotine plasma concentration is substantially below the maximum level obtained during treatment. The preferred length of the discontinuance period depends on the concentration of the effective formulation dosage and the form of the formulation used. The discontinuance period can be at least 2 days, preferably is at least 4 days, more preferably is at least 1 week and generally does not exceed a period of 4 weeks unless the patient has overcome the addiction to nicotine. An intermittent schedule of administration of agent can continue until the desired therapeutic effect, and ultimately treatment of the addition, is achieved.

In yet another embodiment, intermittent administration of the therapeutically effective formulation dose is cyclic. By “cyclic” is intended intermittent administration accompanied by breaks in the administration, with cycles ranging from about 1 week to about 2, 3, 4, 5, or 6 weeks, more preferably about 2 weeks to about 4 weeks. For example, the administration schedule might be intermittent administration of the effective formulation dose with a single dose is given three times per week for 4 weeks, followed by a break in intermittent administration for a period of a week, followed by intermittent administration by administration of a single dose given once per week for 3 weeks, and so forth. A cyclic intermittent schedule of administration of the formulation to a patient may continue until the nicotine addiction is overcome.

### VI. Assessing Addiction

A variety of methods may be utilized to assess the craving for nicotine, including but not limited to, the nicotine craving test specified by the Diagnostic and Statistical Manual of Mental Disorders, Revised Third Edition (DSM-III-R) (see (1991) J. Am. Med. Assoc. 266:3133); the Shiffman-Jarvik Craving Subscale (see O'Connell and Martin (1987) J. Consult. Clin. Psychol. 55:367-371 and Steur and Wewers (1989) ONF 16:193-198, also describing a parallel visual analog test); West et al. (1984) Br. J. Addiction 79:215-219; and Hughes et al. (1984) Psychopharmacology 83:82-87, each of which is expressly incorporated herein by reference.

A preferred nicotine craving scale is that specified in DSM-III-R, supra. According to this scale, a subject is asked to rate the severity of his craving for nicotine on a scale between 0 and 4, wherein 0 is none; 1 is slight; 2 is mild; 3 is moderate; and 4 is severe. Using the compositions and methods described herein, the subject should attain at least a one unit, and preferably at least a two unit, decrease in his craving for nicotine as measured by the protocol set forth in DSM-III-R from about 2 to 30 minutes after administration of the oral nicotine formulation. More preferably, the maximum reduction in craving for nicotine will occur from about 2 to 20 minutes, and more preferably from about 2 to 10 minutes after administration of the oral nicotine formulation.

The Shiffman-Jarvik Craving Scale is a six-item, forced-choice, self-report tool that measures cigarette craving. Each item has seven possible responses which correspond to scores ranging from 1 (no craving) to 7 (high craving). A mean score is obtained to determine the respondent's level of craving. A typical craving score measured 48 hours after the initiation of a smoking cessation program is between about 4 and 5; while a two-week follow-up craving scale will typically be between about 3 and 4. Using the compositions and methods described herein, the subject should attain at least a one unit, and preferably at least a two unit, decrease in his craving for nicotine as measured by the protocol set forth in the Shiffman-Jarvik Craving Scale from about 2 to 30 minutes after administration of the oral nicotine formulation. More preferably, the maximum reduction in craving for nicotine will occur from about 2 to 20 minutes, and more preferably from about 2 to 10 minutes after administration of the oral nicotine formulation.

The “craving questionnaire” craving scale employs a five item questionnaire that asks subjects to rate how much they had been missing their cigarettes, how difficult it had been to be without cigarettes, how much they had been aware of not smoking, how pre-occupied they had been with thinking about cigarettes, and how much they had craved their cigarettes. The subject responds to each question with a number between 1 and 3, where 1 is low and 3 is high. The ratings are combined to give a single craving score. According to this craving scale, a combined score of between about 9 and 12 is typical. Using the compositions and methods described herein, the subject should attain at least a three unit, and preferably at least a four unit, decrease in his craving for nicotine as measured by the protocol set forth for use with this craving questionnaire from about 2 to 30 minutes after administration of the oral nicotine formulation. More preferably, the maximum reduction in craving for nicotine will occur from about 2 to 20 minutes, and more preferably from about 2 to 10 minutes after administration of the oral nicotine formulation.

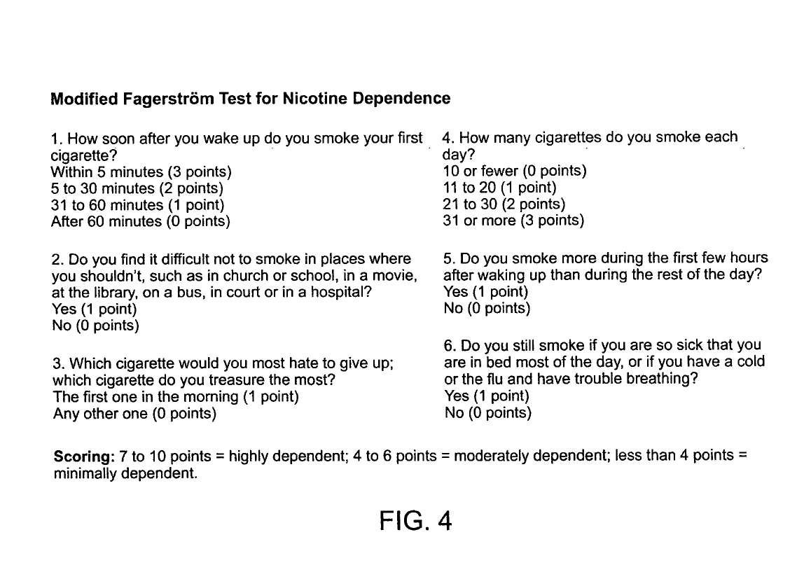

A subject's nicotine dependence can be quantified using an eight-question scale, termed the Fagerstrom Nicotine Tolerance Scale (see Fagerstrom (1978) Addict. Behav. 3:235-241 and Sachs (1986) Clinics in Geriatric Medicine 2:337-362) which provides a relative index of the degree of physical dependency that a patient has for nicotine. This test is shown in FIG. 4.

These tests have a variety of uses in practicing the instant invention. For example, the Fagerstrom test may be used to estimate nicotine tolerance and therefore the initial nicotine dose in treatment. Cravings scores may be used to determine the effectiveness of a given formulation dosage in suppressing the desire to smoke or chew tobacco.

As will be evident to one of skill in the art, the ability to measure the patient's arterial nicotine plasma levels can be of tremendous value in tailoring a smoking cessation or other therapy to the patient's needs. There has been very little discussion in the literature of using direct or indirect measurement of arterial nicotine levels as an integral part of smoking cessation therapy. The traditional interest in quantifying arterial nicotine levels has been related to research on efficacy of smoking cessation therapies. For example, research studies commonly used various measurement techniques to attempt to verify self-reports of smoking frequencies by study subjects. These include the measurement in saliva and blood plasma of nicotine, cotinine (the primary metabolite of nicotine), carboxyhemoglobin, and thiocyanate; and the measurement in expired air of carbon monoxide. The most frequently cited technique is the quantification of cotinine, a nicotine metabolite, in saliva. The quantification of cotinine in blood fluids can be accomplished by gas-liquid chromatography, radioimmunoassay, and liquid chromatography. (For a discussion of liquid chromatographic assays for cotinine, see Machacek and Jiang (1986) Clin. Chem. 32:979-982, herein incorporated by references.)

The present invention may optionally include the direct or indirect measurement of nicotine blood levels as an integral part of methods for treating conditions responsive to nicotine therapy, and particularly for smoking cessation therapy and for reducing nicotine craving. The nicotine blood levels can be measured before, during, or after the administration of the formulations of the invention, as an aid in determining the amount of nicotine to be administered and the frequency of administration. In a preferred embodiment, saliva samples are taken from the patients and used for measurement of cotinine, as a biochemical marker of nicotine blood plasma levels. Cotinine levels are determined using any of the analytical methods known to those skilled in the art. In a particularly preferred embodiment, the cotinine assay would be portable and easily and simply accomplished by the patient, as in an assay kit or strip indicator.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results.

### EXAMPLES

### Example 1

### Single-Dose Application of Deep Lung Nicotine Formulation

Smoking dependence appears partly related to the “high & fast” rise in plasma nicotine concentration achieved by cigarettes. However, unlike cigarettes, current nicotine replacement therapies (NRTs) attain relatively “low & slow” nicotine plasma levels (FIG. 1). This example illustrates that a nicotine delivery system that provides cigarette-like plasma levels, serves to reduce acute craving, inhibit relapse, and result in higher smoking cessation rates compared with existing NRTs.

The AERx Essence System known in the art was used to deliver single-bolus doses of aerosolized nicotine to healthy adult male smokers. The AERx Essence is an all-mechanical, nonpropellent driven, hand-held device that uses individually packaged, single-use, dosage form strips. A uniformly fine, respirable aerosol is created when the drug solution is “extruded” through an array of submicron sized holes drilled into the dosage form strip. The fine aerosol that is generated allows the deep-lung deposition needed to achieve rapid and efficient absorption of drug similar to that obtained by smoking. This inhalation delivery system is a part of the AERx inhalation delivery platforms; AERx devices may be all-mechanical, or electro-mechanical. They may also have various electronic components. Some of these devices include diagnostic and disease management tools as well. The AERx Essence device actuates the piston movement when the patient pushes a button that also causes opening of the valve through which the air that the patient is inhaling enters the device. The inspiratory flow rate is mechanically controlled in this particular embodiment of the AERx Essence device.

Methods

Eighteen healthy, adult male smokers were enrolled in a randomized, open-label, multiple-exposure study which was conducted in two parts. Two subjects were removed prior to Study Part 2 with sixteen subjects starting and completing Study Part 2. Subjects' ages ranged from 19-41 years (mean=27 years).

In Study Part 1, the tolerability and safety of seven nicotine concentrations were evaluated. In Study Part 2, subjects received one of three nicotine concentrations: 10, 20, or 30 mg/ml, delivering bolus nicotine lung doses of approximately 0.2, 0.4 and 0.7 mg, respectively. Measures of arterial nicotine plasma concentration and acute post-dosing cigarette craving scores (11-point VAS) were made following a single inhalation of nicotine.

Results

Safety and Tolerability: No clinically significant changes in safety measures were noted following dosing (vital signs, ECG, spirometry, labs). A total of 119 adverse events (AEs) were recorded. Most AEs were reported as either mild or moderate and self-resolved without medication. No serious AEs were observed. The most commonly reported AEs were throat irritation, lightheadedness (Table 1).

TABLE 1
Incidence of most common Adverse Events (AE)

| Adverse Event (AE) | Incidence | Subjects Experiencing AE |
|---|---|---|
| Throat irritation | 46 | 17 |
| Lightheadedness | 22 | 11 |
| Cough | 20 | 10 |

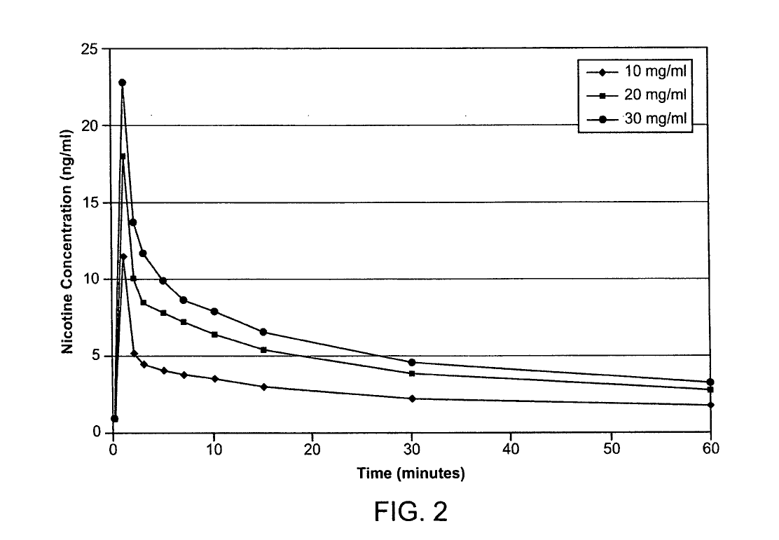

Pharmacokinetics: Arterial plasma nicotine pharmacokinetics demonstrated rapid onset (Tₘₐₓ=1 min) and substantial peak plasma concentrations. Maximum plasma concentrations (Cmax) and area under the concentration-time curves (AUC) were consistent with a trend toward dose proportionality (FIG. 2, Table 2).

TABLE 2
Mean Nicotine Pharmacokinetic Parameters

| Parameter | 10 mg/ml | | 20 mg/ml | | 30 mg/ml | |
|---|---|---|---|---|---|---|
| Tmax (min) | 1 | | 1 | | 1 | |
| Cmax (ng/ml) | 11.5 | (9.5) | 18.0 | (3.6) | 22.9 | (9.0) |
| T½ (min⁻¹) | 136 | (58) | 114 | (18) | 97 | (16) |
| AUC₀₋ₜ (ng · min/ml) | 319 | (219) | 532 | (116) | 622 | (218) |

Standard deviations are in parenthesis.

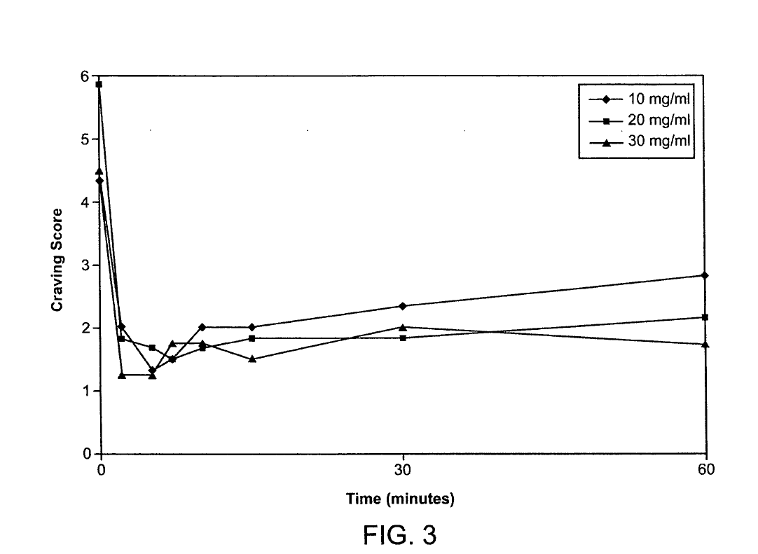

Acute Craving: Patients were asked to rate their nicotine craving on a scale of 0 to 10 pre- and post-dosing. Nearly all subjects reported an acute reduction in craving or an absence of craving immediately following study dosing. A mean reduction in craving from baseline was observed following all three dose levels (FIG. 3). Combining all dose levels, mean craving declined from 4.9 to 1.4 within 5 minutes post-dosing, and remained below pre-dose baseline for the 4 hours of monitoring.

### CONCLUSIONS

Inhaled nicotine via the AERx Essence appears safe and tolerable. The AERx Essence delivers inhaled nicotine with a PK profile that is consistent with the rapid delivery and absorption seen with cigarette smoking, and acute craving following inhaled nicotine via the AERx Essence appears to be acutely reduced

### Example 2

### Use of Alternative Nicotine Forms

This example demonstrates the effectiveness of different nicotine dosage forms of the invention. The aim of the example is to illustrate that generically available nicotine formulations are suitable for use in the present invention.

Formulation studies were performed to evaluate the effectiveness of nicotine salts and pH on the stability of nicotine in AERx® dosage forms. Nicotine is a weak base (pKa₁=3.4 and pKa₂=8.4) and in the un-ionized state had the capability to get absorbed into the polymeric materials used in many nicotine delivery systems. When a screening study was conducted in the pH 3.0-7.0 range using buffered nicotine sulphate and bitartrate, nicotine concentration was in effect unaltered for the two salts at the lower pH's of 3.0 and 4.0. Nicotine bitartrate was better in this pH range as compared to nicotine sulphate in terms of ensuring that there was no loss of nicotine into the polymeric dosage form materials. A theoretical calculation using the Henderson-Hasselbach equation indicated that the ratio of ionized to un-ionized species at pH 3.0 and pH 4.0 was 158489 and 15849, respectively, implying limited potential for absorption to occur at the lower pH of 3.0.

Aradigm's proprietary AERx® System was used in the present example. This system consists of the AERx® Strip™, a single-use disposable dosage form, and the AERx® device, which has two hand-held configurations: an electromechanical version and an all-mechanical version.

Nicotine formulations were packaged under aseptic conditions into the AERx® Strip, to create a sterile dosage form. Aerosol generation using the AERx® System is completed in one or two seconds via mechanical pressurization of the nicotine formulation. This pressurization causes the seal in the AERx® Strip between the drug reservoir and a nozzle array to peel open. This leads to the nicotine formulation being expelled through the nozzle array as a fine aerosol. By varying the size of the nozzle holes, the size of the aerosol can be modified to optimize regional lung deposition. The electromechanical AERx® system was modified to allow addition of dose titration capabilities into the system for this program.

Results

Analytical Assay Development for Nicotine Quantitation

A high performance liquid chromatography (HPLC)-based assay was developed in house to enable quantitation of nicotine (Table 3). The HPLC method was suitably modified for functional (aerosol) testing of AERx®-nicotine and a partial qualification conducted. The analytical performance parameters evaluated were: standard linearity, range, accuracy, precision, limit of quantitation (LOQ), system suitability, specificity and solution stability. The functional test method, in conjunction with the RP-HPLC method was qualified for use in determining emitted dose and particle size distribution of aerosolized nicotine. Nicotine working standard linearity, r2, was 1.000 and the linear concentration range was 0.5 to 40.0 μg/mL (Table 4).

TABLE 3
Analytical Method Parameters/Details
Reverse Phase High Performance Liquid
Chromatography (RP-HPLC) Method

| HPLC Column | Ace 5, C18 (25 cm × 4.6 mm, 5 μm) | |
|---|---|---|
| Mobile Phase | 80% 20 mM Phosphate buffer, | |
| | 20% Methanol, pH 5.0 | |
| Wavelength (UV detector) | 259 | nm |
| Flow Rate | 1.00 | mL/min |
| Injection Volume | 20 | μL |
| Column Temperature | 35° | C. |
| Autosampler Temperature | Ambient | |
| Run Time | 10 | minutes |

TABLE 4
Summary of analytical results from method development

| Analytical Performance | |
|---|---|
| Parameters Evaluated | Results |
| Standard Linearity and Range | R² = 1.000, 0.5-40.0 μgmL |
| Accuracy and Precision | Passed acceptance criteria |
| Limit of Quantitation | 0.5 μg/mL |
| System Suitability and | Peak Area & RT: % RSD < 2%, Tailing |
| Specificity | Factor = 1.0, No interfering peak |
| Solution Stability | Standards Stability = 7 days, |
| | Diluent/Mobile Phase Stability = 15 days |

Nicotine Formulation Development

Selecting Nicotine salts: After evaluation of availability of various grades of nicotine salts on the market, nicotine bitartrate and nicotine sulphate were selected for further screening. Both salts were purchased from Nicobrand Limited, Northern Ireland.

Formulation Concentrations: A 0.9-1.0 mg lung dose was estimated as an efficacious upper end dose based on available literature. Estimating a 60% deep lung delivery efficiency for AERx®, the nicotine concentration chosen at the upper end was 32.0 mg/mL. Using the three step dose reduction strategy described above, the lower nicotine concentration was estimated to be 10.7 mg/mL. Initial formulation studies used a lower concentration of 8.0 mg/mL (prior to the finalization of a three-step dose reduction strategy), which was later finalized (using a three step dose reduction strategy) to be 10.7 mg/mL.

Formulation stability in pouches: An initial formulation screening study was initiated utilizing nicotine formulations between the pH of 3.0-7.0 stored in pouches at 40° C./75% R.H. The pouches were made of the same polymeric material as the contact layer in AERx® dosage forms. In previous studies with a different but chemically similar drug, polymeric materials showed the potential for absorptive losses of drug from solution. Nicotine concentration as well as pH was monitored for a period of 28 days.

Results indicated no impact on pH over the 28 days period throughout the pH range evaluated (Tables 5 & 6). The concentration of nicotine decreased over time at higher pH values, consistent with the proposed absorption when in the unionized form (Tables 7 & 8). The concentration of nicotine was unaltered at pH's 3.0 and 4.0.

TABLE 5
Formulation stability- nicotine bitartrate pH results in pouches

| | Nicotine Bitartrate (controls) | | | | | | | | Nicotine Bitartrate (pouches) | | | | | | | |
|---|---|---|---|---|---|---|---|---|---|---|---|---|---|---|---|---|
| | 8 mg/mL | | | | 32 mg/mL | | | | 8 mg/mL | | | | 32 mg/mL | | | |
| Theoretical | | T = 7 | T = 15 | T = 28 | | T = 7 | T = 15 | T = 28 | | T = 7 | T = 15 | T = 28 | | T = 7 | T = 15 | T = 28 |
| pH | T = 0 | days | days | days | T = 0 | days | days | days | T = 0 | days | days | days | T = 0 | days | days | days |
| 3.0 | 3.0 | 3.1 | 3.1 | 3.1 | 3.0 | 3.1 | 3.1 | 3.1 | 3.1 | 3.1 | 3.3 | 3.2 | 3.1 | 3.1 | 3.2 | 3.2 |
| 4.0 | 4.0 | 4.3 | 4.2 | 4.2 | 4.0 | 4.3 | 4.3 | 4.3 | 4.3 | 4.2 | 4.3 | 4.4 | 4.3 | 4.2 | 4.3 | 4.4 |
| 5.0 | 5.1 | 5.0 | 5.3 | 5.3 | 5.0 | 5.3 | 5.3 | 5.3 | 5.4 | 5.3 | 5.2 | 5.2 | 5.4 | 5.3 | 5.2 | 5.2 |
| 6.0 | 6.0 | 6.3 | 6.1 | 6.2 | 6.0 | 6.3 | 6.2 | 6.2 | 6.3 | 6.2 | 6.1 | 6.1 | 6.3 | 6.2 | 6.0 | 6.1 |
| 7.0 | 7.1 | 7.1 | 7.4 | 7.3 | 7.0 | 7.1 | 7.2 | 7.2 | 7.4 | 7.1 | 7.1 | 7.1 | 7.2 | 6.9 | 7.0 | 6.8 |

TABLE 6
Formulation stability- nicotine sulphate pH results in pouches

| | Nicotine Sulphate (controls) | | | | | | | | Nicotine Sulphate (pouches) | | | | | | | |
|---|---|---|---|---|---|---|---|---|---|---|---|---|---|---|---|---|
| | 8 mg/mL | | | | 32 mg/mL | | | | 8 mg/mL | | | | 32 mg/mL | | | |
| Theoretical | | T = 7 | T = 15 | T = 28 | | T = 7 | T = 15 | T = 28 | | T = 7 | T = 15 | T = 28 | | T = 7 | T = 15 | T = 28 |
| pH | T = 0 | days | days | days | T = 0 | days | days | days | T = 0 | days | days | days | T = 0 | days | days | days |
| 3.0 | 3.0 | 2.7 | 2.8 | 2.8 | 3.0 | 2.7 | 2.8 | 2.8 | 2.9 | 2.7 | 2.8 | 2.8 | 2.8 | 2.8 | 2.9 | 2.8 |
| 4.0 | 4.0 | 4.1 | 4.1 | 4.1 | 4.1 | 4.0 | 4.0 | 4.1 | 4.1 | 4.0 | 4.1 | 4.1 | 4.0 | 4.0 | 4.1 | 4.1 |
| 5.0 | 5.0 | 5.1 | 5.1 | 5.1 | 5.1 | 5.1 | 5.1 | 5.1 | 5.2 | 5.1 | 5.0 | 5.1 | 5.2 | 5.1 | 5.0 | 5.0 |
| 6.0 | 6.0 | 6.2 | 6.1 | 6.1 | 6.0 | 6.2 | 6.1 | 6.1 | 6.2 | 6.1 | 6.0 | 6.1 | 6.2 | 6.0 | 5.9 | 6.0 |
| 7.0 | 7.0 | 7.3 | 7.1 | 7.1 | 7.0 | 7.2 | 7.1 | 7.1 | 7.1 | 6.9 | 6.9 | 6.9 | 7.1 | 6.9 | 6.9 | 6.8 |

TABLE 7
Formulation stability- nicotine bitartrate concentration results in pouches

| | Nicotine Bitartrate | | | | | | | |
|---|---|---|---|---|---|---|---|---|
| Formulation | % Recovery (8 mg/mL) | | | | % Recovery (32 mg/mL) | | | |
| Theoretical pH | T = 0 | T = 7 days | T = 15 days | T = 28 days | T = 0 | T = 7 days | T = 15 days | T = 28 days |
| 3.0 | 101.4 | 99.9 | 100.0 | 101.6 | 101.7 | 99.1 | 101.9 | 102.3 |
| 4.0 | 100.8 | 99 | 99.0 | 100.2 | 100.5 | 101.9 | 99.1 | 98.7 |
| 5.0 | 100.6 | 96.8 | 97.1 | 98.3 | 100.5 | 99.2 | 95.4 | 98.9 |
| 6.0 | 101.7 | 93.2 | 90.1 | 95.8 | 99.2 | 93.1 | 94.7 | 95.3 |
| 7.0 | 97.6 | 72.3 | 73.5 | 75.0 | 98.6 | 85.8 | 89.3 | 87.3 |

TABLE 8
Formulation stability- nicotine sulphate concentration results in pouches

| | Nicotine Sulphate | | | | | | | |
|---|---|---|---|---|---|---|---|---|
| Formulation | % Recovery (8 mg/mL) | | | | % Recovery (32 mg/mL) | | | |
| Theoretical pH | T = 0 | T = 7 days | T = 15 days | T = 28 days | T = 0 | T = 7 days | T = 15 days | T = 28 days |
| 3.0 | 100.3 | 94.9 | 96.9 | 102.4 | 98.1 | 101.2 | 100.2 | 99.0 |
| 4.0 | 99.5 | 95.4 | 98.3 | 100.9 | 98.4 | 97.0 | 98.3 | 99.0 |
| 5.0 | 100.4 | 95.6 | 95.2 | 98.5 | 98.5 | 97.8 | 99.0 | 98.0 |
| 6.0 | 100.2 | 94.3 | 92.5 | 94.0 | 99.3 | 94.6 | 95.0 | 95.8 |
| 7.0 | 97.6 | 75.0 | 75.2 | 82.2 | 99.0 | 89.6 | 89.4 | 90.1 |

Based on these results as well as theoretical calculations, pH 3.0 was chosen for use with polymeric products as the proportion of ionized species is maximized at this pH while maintaining acceptable safety profiles for an inhaled product.

Formulation stability/screening in AERx® dosage forms: As buffering at extreme pH's is not desirable for inhaled products because it can elicit hyperreactivity, pH adjustment is preferred. For this reason an unbuffered formulation was evaluated.

AERx® dosage forms were filled with nicotine bitartrate and nicotine sulphate at both 10.7 and 32.0 mg/mL of nicotine and stored at 40° C./15% R.H. (accelerated storage condition recommended for semi-permeable containers, ICH Q1A) for a period of 14 days.

The results for pH (Table 9) and concentration (Table 10) indicated excellent control, confirming the choice of an unbuffered formulation. Having developed a robust formulation, we then proceeded to evaluate the dose titration capabilities as well as optimizing aerosol performance using these formulations.

TABLE 9
Nicotine in AERx ® strips (stored at 40° C./15% RH)

| | pH values | | |
|---|---|---|---|
| Formulation | T = Initial | T = 7 days | T = 14 days |
| Nicotine Bitartrate | 3.0 | 2.9 | 2.9 |
| (10.7 mg/mL, pH 3.0) | | | |
| Nicotine Bitartrate | 3.0 | 3.0 | 2.9 |
| (32.0 mg/mL, pH 3.0) | | | |
| Nicotine Sulphate | 3.0 | 2.9 | 2.9 |
| (10.7 mg/mL, pH 3.0) | | | |
| Nicotine Sulphate | 3.0 | 2.9 | 2.9 |
| (32.0 mg/mL, pH 3.0) | | | |

TABLE 10
Recovery of nicotine in AERx ® strips stored at 40° C./15% RH

| | % Recovery (SD) | | | | |
|---|---|---|---|---|---|
| Formulation | T = Initial | | T = 7 days | T = 14 days | |
| Nicotine Sulphate | 98.9 | (0.5) | 102.1 (0.3) | 99.3 | (0.2) |
| (10.7 mg/mL, pH 3.0) | | | | | |
| Nicotine Sulphate | 100.2 | (0.8) | 100.2 (0.5) | 102.9 | (6.5) |
| (32.0 mg/mL, pH 3.0) | | | | | |
| Nicotine Sulphate | 100.0 | (0.4) | 100.5 (0.2) | 100.7 | (1.1) |
| (10.7 mg/mL, pH 3.0) | | | | | |
| Nicotine Sulphate | 97.4 | (2.3) | 100.6 (0.9) | 100.0 | (0.7) |
| (32.0 mg/mL, pH 3.0) | | | | | |

Optimization of Aerosol Performance of Nicotine Formulation with the AERx® System

Characterization and optimization of delivery efficiency (emitted dose) of nicotine formulations from AERx® in a simulated inhalation:

Efficiency of delivery of formulation from the AERx® System is expressed as emitted dose (ED). For ED quantification, a known dose of each nicotine formulation was loaded into AERx® Strips and then aerosolized onto standardized collection filters. The filters were rinsed thoroughly with the assay diluent. Spiking studies were conducted to verify that all of the nicotine was recovered from the filter. The amount of nicotine in the rinsate was quantified by HPLC.

The ED data was excellent for the partial extrusion as well as multiple concentrations dose reduction strategies evaluated. Emitted dose in percent at the three levels using the partial extrusion strategy was 20.4, 17.2 and 18.8 with standard deviations of 1.4, 0.8 and 1.0 respectively (see Table 11). The percent emitted dose for the successive concentrations of 32.0, 21.3 and 10.7 mg/mL was 60.0, 61.7 and 62.7 with the standard deviations being 3.0, 2.8 and 3.2 respectively (see Table 12).

TABLE 11
Emitted dose performance using partial dose
settings using 32 mg/mL nicotine bitartrate

| | Level 1 | Level 2 | Level 3 | | | % 2nd | % 3rd |
|---|---|---|---|---|---|---|---|
| DF# | ED (% LC) | ED (% LC) | ED (% LC) | Total ED | % 1st shot | shot | shot |
| 1 | 18.7 | 14.7 | 18.7 | 52.2 | 35.8 | 28.3 | 35.9 |
| 2 | 20.8 | 17.1 | 20.2 | 58.1 | 35.8 | 29.4 | 34.8 |
| 3 | 18.3 | 17.2 | 18.8 | 54.2 | 33.7 | 31.7 | 34.6 |
| 4 | 19.9 | 17.1 | 18.8 | 55.8 | 35.7 | 30.6 | 33.7 |
| 5 | 21.2 | 17.2 | 20.0 | 58.4 | 36.3 | 29.4 | 34.2 |
| 6 | 20.6 | 18.2 | 19.3 | 58.1 | 35.4 | 31.4 | 33.2 |
| 7 | 19.9 | 17.7 | 18.3 | 55.8 | 35.6 | 31.6 | 32.8 |
| 8 | 18.3 | 17.5 | 18.3 | 54.2 | 33.8 | 32.3 | 33.9 |
| 9 | 22.8 | 17.4 | 19.5 | 59.8 | 38.2 | 29.1 | 32.6 |
| 10 | 20.6 | 17.6 | 19.4 | 57.7 | 35.7 | 30.6 | 33.7 |
| 11 | 20.3 | 17.0 | 19.1 | 56.4 | 36.1 | 30.1 | 33.8 |
| 12 | 22.0 | 17.1 | 20.5 | 59.6 | 37.0 | 28.6 | 34.4 |
| 13 | 20.6 | 17.4 | 17.8 | 55.8 | 36.9 | 31.2 | 31.9 |
| 14 | 21.0 | 17.1 | 17.7 | 55.8 | 37.6 | 30.7 | 31.7 |
| 15 | 20.4 | 17.7 | 18.8 | 56.9 | 35.8 | 31.1 | 33.1 |
| 16 | 20.3 | 16.9 | 17.3 | 54.5 | 37.3 | 31.0 | 31.7 |
| 17 | 22.2 | 17.3 | 17.9 | 57.4 | 38.6 | 30.1 | 31.2 |
| 18 | 21.6 | 18.3 | 18.7 | 58.7 | 36.9 | 31.2 | 31.9 |
| 19 | 20.2 | 17.8 | 20.5 | 58.4 | 34.6 | 30.4 | 35.0 |
| 20 | 17.5 | 15.4 | 16.8 | 49.7 | 35.2 | 31.0 | 33.7 |
| Mean | 20.4 | 17.2 | 18.8 | 56.4 | 36.1 | 30.5 | 33.4 |
| SD | 1.36 | 0.82 | 1.03 | 2.53 | 1.29 | 1.07 | 1.27 |

TABLE 12
Emitted dose performance of nicotine formulations at various concentrations

| | Full Extrusion: ED (% LC) | | | Full Extrusion: ED (mg) | | |
|---|---|---|---|---|---|---|
| | 32.0 mg/mL | 21.3 mg/mL | 10.7 mg/mL | 32.0 mg/mL | 21.3 mg/mL | 10.7 mg/mL |
| | Nicotine | Nicotine | Nicotine | Nicotine | Nicotine | Nicotine |
| ED # | Bitartrate | Bitartrate | Bitartrate | Bitartrate | Bitartrate | Bitartrate |
| 1 | 56.2 | 55.6 | 59.5 | 0.90 | 0.59 | 0.32 |
| 2 | 59.4 | 59.7 | 62.6 | 0.95 | 0.64 | 0.34 |
| 3 | 56.9 | 61.8 | 60.4 | 0.91 | 0.66 | 0.32 |
| 4 | 56.6 | 60.4 | 62.9 | 0.91 | 0.64 | 0.34 |
| 5 | 57.7 | 60.9 | 65.3 | 0.92 | 0.65 | 0.35 |
| 6 | 58.1 | 64.8 | 62.3 | 0.93 | 0.69 | 0.33 |
| 7 | 63.6 | 57.9 | 60.0 | 1.02 | 0.62 | 0.32 |
| 8 | 60.3 | 65.2 | 58.1 | 0.96 | 0.69 | 0.31 |
| 9 | 54.6 | 59.9 | 66.1 | 0.87 | 0.64 | 0.35 |
| 10 | 58.4 | 58.0 | 67.9 | 0.93 | 0.62 | 0.36 |
| 11 | 59.8 | 60.1 | 66.8 | 0.96 | 0.64 | 0.36 |
| 12 | 63.8 | 63.0 | 61.8 | 1.02 | 0.67 | 0.33 |
| 13 | 60.0 | 59.7 | 64.6 | 0.96 | 0.64 | 0.35 |
| 14 | 63.1 | 62.8 | 65.0 | 1.01 | 0.67 | 0.35 |
| 15 | 61.2 | 63.7 | 56.0 | 0.98 | 0.68 | 0.30 |
| 16 | 64.5 | 66.0 | 62.0 | 1.03 | 0.70 | 0.33 |
| 17 | 62.7 | 62.0 | 65.2 | 1.00 | 0.66 | 0.35 |
| 18 | 65.5 | 65.7 | 63.7 | 1.05 | 0.70 | 0.34 |
| 19 | 58.1 | 64.2 | 65.1 | 0.93 | 0.68 | 0.35 |
| 20 | 59.9 | 62.0 | 58.0 | 0.96 | 0.66 | 0.31 |
| Mean | 60.0 | 61.7 | 62.7 | 0.96 | 0.66 | 0.34 |
| SD | 3.0 | 2.8 | 3.2 | 0.05 | 0.03 | 0.02 |
| % RSD | 5.1 | 4.6 | 5.1 | 5.1 | 4.6 | 5.1 |

Development of Dose-Titration Capabilities

Partial Extrusion of a Single AERx® Strip

Partial extrusion of an AERx® Strip was carried out by altering the settings for the piston position, to program it to aerosolize only a portion of the contents of the AERx® Strip. Testing was done using nicotine formulations, with the results being presented in Table 11. The delivered dose in percent of emitted dose at the three levels was 36.1, 30.5 and 33.4 with standard deviations of 1.3, 1.1 and 1.3 respectively. This corresponds to a nicotine dose of 0.33 mg, 0.28 mg and 0.30 mg at the three dose levels respectively.

Altering the Concentration of Nicotine in AERx® Strip

The emitted dose and particle size distribution of nicotine formulations at various concentrations was evaluated. In order to keep the delivered dose constant, the range of concentrations tested were matched to the results of the aerosol performance studies from partial extrusion discussed above. Results are presented in Table 13. The percent emitted dose for the successive concentrations of 32.0, 21.3 and 10.7 mg/mL was 60.0, 61.7 and 62.7 with the standard deviations being 3.0, 2.8 and 3.2 respectively. The corresponding delivered nicotine dose at the three concentrations was calculated to be 0.96 mg, 0.66 mg and 0.34 mg with standard deviations of 0.05, 0.03 and 0.02 respectively.

TABLE 13
Emitted dose summary

| | Type of Extrustion | | Emitted Drug | Dose to the lung |
|---|---|---|---|---|
| Formulation | (N = 20) | % ED (SD) | (mg) | [FPF_{3.5} = 0.78] (mg) |
| 32.0 mg/mL Nicotine | Partial Dose Level 1 | 20.4 (1.36) | 0.33 | 0.26 |
| Bitartrate, pH 3.0 | Partial Dose Level 2 | 37.9 (1.96) | 0.61 | 0.48 |
| | Partial Dose Level 3 | 56.4 (2.53) | 0.90 | 0.71 |
| 10.7 mg/mL Nicotine | Full Extrusion | 62.7 (3.22) | 0.34 | 0.27 |
| Bitartrate, pH 3.0 | | | | |
| 21.3 mg/mL Nicotine | Full Extrusion | 61.7 (2.82) | 0.66 | 0.51 |
| Bitartrate, pH 3.0 | | | | |
| 32.0 mg/mL Nicotine | Full Extrusion | 60.0 (3.04) | 0.96 | 0.75 |
| Bitartrate, pH 3.0 | | | | |

Optimizing Particle Size Distribution of the Aerosol Droplets of Nicotine Formulations Generated Using AERx®

Particle size distribution (PSD) is a key determinant of the regional lung deposition of inhaled aerosols. A cascade impactor (Series 20-800 Mark II, Thermo Andersen), which size selectively collects the aerosol by inertial impaction on a series of stages, was used to characterize the aerosol PSD. The PSD was characterized in terms of Mass Median Aerodynamic Diameter (MMAD) and Geometric Standard Deviation (σg). MMAD denotes the particle size at which half of the total aerosol mass is contained in larger particles and half in smaller particles. The σg indicates the variability of aerosol particle sizes. An aerosol composed of identical size particles would have a σg of 1.0; σg of ≦1.3 is considered monodisperse; σg of ≧1.3 is considered polydisperse.

We evaluated PSD with the optimized nicotine formulations. The MMAD ranged between 2.5-2.7 μm for the different combinations of device and formulation combinations (see Table 14). The GSD was 1.3, which indicates the monodispersity of the aerosol. The influence of PSD on nicotine kinetics, efficiency, and success rates with the product would need to be determined as part of the Phase II proposal. The fraction of particles under 3.5 μm is typically used to evaluate the fraction of aerosol capable of deposition in the deep lung. The typical fine particle fraction was about 80% (Table 14), indicating that the majority of the deposited aerosol was capable of deep lung deposition, key to the success of the therapy.

TABLE 14
Particle Size Distribution (PSD) summary

| | Type of | | | |
|---|---|---|---|---|
| | extrusion | | | |
| Formulation | (N = 3) | MMAD (SD) | GSD (SD) | FPF_{8.6} |
| 32.0 mg/ml Nicotine | 3 partial | 2.66 (0.04) | 1.28 (0.01) | 0.779 |
| Bitartrate, pH 3.0 | extrusions | | | |
| 32.0 mg/ml Nicotine | 1 partial | 2.65 (0.03) | 1.28 (0.01) | 0.783 |
| Bitartrate, pH 3.0 | extrusion | | | |
| 32.0 mg/ml Nicotine | Full | 2.49 (0.04) | 1.35 (0.02) | 0.762 |
| Bitartrate, pH 3.0 | extrusions | | | |

Characterization of Stability of Nicotine Formulations in AERx® Strip Dosage Forms

In the following set of experiments, the stability of the selected nicotine formulations was evaluated in AERx® Strip dosage forms.

Physical and chemical characterization of the selected formulations and aerosol performance upon storage in AERx® Strips for up to 1 month:

The primary storage condition for the strips was chosen to be 25° C./40% R.H., as the formulation selected was quite simple and did not require refrigerated storage. The strips were loaded with 50 μL of nicotine formulation, sealed and stored at 25° C./40% R.H., as well as at the accelerated storage condition of 40° C./15% R.H. for up to 1 month. The formulations in the strips were characterized for concentration, pH and content uniformity, in addition to measurement of aerosol performance (emitted dose, particle size distribution) with the AERx® Strips in storage. The results from the one month stability study indicated maintenance of pH, concentration, as well as aerosol performance over the tested stability duration at the primary as well as accelerated storage condition (Tables 15 & 16). The emitted dose (ED) performance at both the concentrations was within normal variability. The MMAD was 2.4 and 2.8 to 2.9 for the two formulation strengths respectively; with GSD's of 1.3, indicating the monodispersity of the aerosol. The fine particle fraction under 3.5 μm was about 82% for the 10.7 mg/mL formulation and 72% for the 32.0 mg/mL formulation. A high fraction of the emitted aerosol in the respirable range ensures that majority of the aerosol will result in deep lung deposition. The data indicates acceptable stability of the formulations in AERx® strips for the duration of the stability study. In the next part of the development program, it will be important to finalize the final formulation concentrations (dependent on the chosen dose reduction and commercialization strategy) and generate stability data to support any proposed clinical studies.

TABLE 15
Summary for Nicotine Bitartrate, 10.7 mg/ml in Aerx ® strips

| | | | T = 4 weeks | | | |
|---|---|---|---|---|---|---|
| Test Attributes | T = Initial | | 25° C./40% RH | | 40° C./15% RH | |
| pH (% RSD) | 3.0 | (0.2) | 3.1 | (0.0) | 3.2 | (0.4) |
| Concentration, mg/mL (% RSD) | 10.8 | (0.8) | 10.7 | (2.1) | 10.7 | (0.3) |
| Content Uniformity Range, | 97.0-100.0 | (1.2) | N/A | | N/A | |
| % LC (% RSD) | | | | | | |
| Unit Dose, % LC (% RSD) | 99.1 | (1.2) | 96.7 | (1.7) | 96.5 | (1.5) |
| Emitted Dose, % LC (% RSD) | 55.1 | (4.9) | 52.0 | (5.8) | 52.5 | (3.0) |
| Emitted Dose Uniformity, % | 95.2-107.0 | (4.9) | 92.7-106.7 | (5.8) | 95.5-102.9 | (3.0) |
| Mean ED (% RSD) | | | | | | |

| | | | |
|---|---|---|---|
| Particle Size Distribution | 2.41, 1.31, 0.81, 44.6 | 2.42, 1.27, 0.85, 44.2 | 2.43, 1.28, 0.82, 43.1 |
| [Record: MMAD (μm), GSD, | | | |
| FPF_{3.5}, FPD (% LC)] | | | |

TABLE 16
Stability Summary for Nicotine Bitartrate, 32.0 mg/mL in AERx ® strips

| | | | T = 4 weeks | | | |
|---|---|---|---|---|---|---|
| Test Attributes | T = Initial | | 25° C./40% RH | | 40° C./15% RH | |
| pH (% RSD) | 3.0 | (0.0) | 3.1 | (0.5) | 3.1 | (0.8) |
| Concentration, mg/mL (% RSD) | 32.5 | (0.9) | 31.2 | (1.0) | 31.5 | (1.0) |
| Content Uniformity Range, % LC | 99.2-101.5 | (0.8) | N/A | | N/A | |
| (% RSD) | | | | | | |
| Unit Dose, % LC (% RSD) | 100.3 | (0.8) | 96.7 | (0.7) | 96.9 | (0.5) |
| Emitted Dose, % LC (% RSD) | 58.3 | (4.3) | 52.8 | (3.1) | 55.9 | (1.3) |
| Emitted Dose Uniformity, % Mean | 94.0-106.8 | (4.3) | 95.6-103.1 | (3.1) | 97.9-101.5 | (1.3) |
| ED (% RSD) | | | | | | |

| | | | |
|---|---|---|---|
| Particle Size Distribution [Record: | 2.77, 1.27, 0.74, 43.1 | 2.87, 1.25, 0.70, 37.0 | 2.87, 1.25, 0.71, 39.7 |
| MMAD (÷m), GSD, FPF_{3.5}, FPD | | | |
| (% LC)] | | | |

### CONCLUSION

The example above supports the feasibility of delivery of nicotine for smoking cessation using the AERx® System with an aqueous formulation that was stable at room temperature for a period of at least a month (duration of stability study). The typical MMAD of the aerosols using either dose reduction strategy was 2.6 μm, whereas the GSD was 1.3. The fine particle fraction was 80%, ensuring deposition of the majority of the emitted aerosol in the deep lung, mimicking smoking, and important for a successful smoking cessation product.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A tobacco-less composition comprising a pharmaceutically active nicotine formulation for delivery to a patient, the nicotine formulation having at least a first and a second form of nicotine and;
the first form of nicotine is present in an amount to provide a first nicotine arterial concentration of at least about 10 ng/ml in the patient within 5 minutes of delivery; and,
the second form of nicotine is present in an amount to maintain a second nicotine arterial concentration of at least about 5 ng/ml in the patient for at least 60 minutes after delivery.

2. The composition of claim 1, further comprising an antidepressant or an anxiolytic

3. The composition of claim 1, wherein the second form of nicotine is encapsulated in a microsphere having a second diameter.

4. The composition of claim 3, wherein the microsphere includes a bioadhesive component.

5. The composition of claim 3, wherein the microsphere is a liposome.

6. The composition of claim 5, wherein the liposome comprises a cyclodextrin.

7. The composition of claim 3, wherein the microsphere is a polyglycolide microsphere.

8. The composition of claim 1, wherein the second form of nicotine comprises a slow release component.

9. The composition of claim 1, wherein the first form of nicotine is encapsulated.

10. The composition of claim 9, wherein the first form of nicotine is substantially free base nicotine.

11. The composition of claim 1, wherein the pharmaceutically active nicotine formulation is a powder, a liquid, a suspension or an emulsion.

12. The composition of claim 11, wherein the pharmaceutically active nicotine formulation is suitable for forming an aerosol.

13. The composition of claim 12, further comprising a propellant suitable for aerosolizing the pharmaceutically active nicotine formulations.

14. The composition of claim 11, wherein the pharmaceutically active nicotine formulation is in the form of a powder and the first form of nicotine has a smaller particle diameter than the second form of nicotine.

15. The composition of claim 14, wherein the first form of nicotine has a particle diameter between about 1 μm and about 4 μm.

16. The composition of claim 14, wherein the second form of nicotine has a particle diameter between about 4 μm and about 12 μm

17. An aerosolized nicotine composition comprising:
a first form of nicotine;
a second form of nicotine; and,
an antidepressant or anxiolytic compound.

18. The aerosolized composition of claim 17, wherein the second form of nicotine is encapsulated in a liposome having a second diameter.

19. The aerosolized composition of claim 18, wherein the first form of nicotine is encapsulated in a liposome having a first diameter that is smaller than the second diameter.
